(19) United States
(12) Patent Application Publication
(10) Pub. No.: US 2023/0277475 A1
(43) Pub. Date: Sep. 7, 2023

(54) BIOACTIVE DECELLULARIZED STEM CELL SHEET FOR TISSUE REPAIR

(71) Applicant: The Chinese University of Hong Kong, Hong Kong (CN)

(72) Inventors: Po Yee Pauline LUI, Hong Kong (CN); Shiyi YAO, Hong Kong (CN); Shu Hang Patrick YUNG, Hong Kong (CN)

(21) Appl. No.: 18/169,958

(22) Filed: Feb. 16, 2023

Related U.S. Application Data
(60) Provisional application No. 63/268,344, filed on Feb. 22, 2022.

(51) Int. Cl. A61K 9/70; C12N 5/0775; A61K 38/18; A61K 38/39; A61K 47/34; A61K 9/00; A61L 27/36; A61L 27/26; A61L 27/60; A61L 27/38

(52) U.S. Cl. CPC A61K 9/7007; A61K 9/0024; A61K 38/1866; A61K 38/1875; A61K 38/39; A61K 47/34; A61L 27/26; A61L 27/3633; A61L 27/3687; A61L 27/3834; A61L 27/60; C12N 5/0662; A61L 2300/252; A61L 2300/258; A61L 2300/414; A61L 2430/02; A61L 2430/06; A61L 2430/10; A61L 2430/30; A61L 2430/40; C12N 2513/00; C12N 2533/40; C12N 2533/54; C12N 2533/90; C12N 2537/00

(57) ABSTRACT

The subject invention pertains to a decellularized stem cell sheet and compositions thereof with retained biological activity. The present invention further relates to the optimized method of producing the decellularized stem cell sheet and methods of using the decellularized stem cell sheet for the promotion of tissue repair in a subject.

### CROSS-REFERENCE TO RELATED APPLICATION

This application claims the benefit of U.S. Provisional Pat. Application Serial No. 63/268,344, filed Feb. 22, 2022, which is hereby incorporated by reference in its entirety including any tables, figures, or drawings.

### BACKGROUND OF THE INVENTION

Both osseointegration at the graft-tunnel interface and remodeling of graft mid-substance (collectively called graft healing) are slow processes after anterior cruciate ligament reconstruction (ACLR)^{14,60}. Biological augmentations are employed for the promotion of graft healing²⁴. Among these, there is an interest in the use of mesenchymal stromal cells (MSCs) for improving ACL graft healing due to their growth-promoting, immunomodulatory and angiogenic effects⁵⁵.

Considering the biocompatibility of scaffolds and inhomogeneous injection of cells, the cell sheet approach is developing rapidly^{25,39,41}. The connective tissue growth factor (CTGF) and ascorbic acid (VitC) treated-tendon-derived stem cell (TDSC) sheet has been shown to promote tendon healing after acute injury and graft healing after ACLR in animal models^{37,38}. However, the need to maintain cell viability and stability, as well as the potential uncontrolled actions of transplanted cells are key issues to be solved for the clinical translation of all stem cell-based therapies¹³. Although MSCs are generally shown to have low immunogenicity and are suitable for allogenic transplantation, tumour and ectopic bone formation have been reported^{1,6,22,53}. The manufacturing cost of MSCs as an advanced therapy product (ATP) and the potential safety concern of stem cell-based therapies by the public delay its clinical translation.

Numerous studies have shown that the growth-promoting, immunomodulatory and angiogenic effects of MSCs in tissue regeneration are mainly mediated by paracrine mechanisms²⁸. Immunomodulatory, chemotactic, and cellular programming factors secreted by MSCs can be retained in the extracellular matrix (ECM) after decellularization^{47,61}. The transplantation of natural decellularized ECM scaffolds alone has been reported to promote tissue repair in various studies, suggesting that decellularized ECM scaffolds possess biological activities even without the cellular component^{40,42}. In addition, the decellularization process also removes cell membrane receptors, which greatly reduces antigenicity.

Musculoskeletal and connective tissue injuries and disorders are common and disabling, presenting devastating impacts to the society. The outcome of musculoskeletal tissue repair is often complicated by fibrous tissue formation, slow healing, and failed healing, particularly in anterior cruciate ligament reconstruction (ACLR), which the outcome of graft healing is poor. Tissue engineering is a potential strategy for the promotion of musculoskeletal tissue repair, including tendon graft healing, after surgery. Our previous study has shown that tenogenic tendon-derived stem cell (TDSC) sheet augmented graft healing after ACLR³⁷. Recent studies have shown that natural extracellular matrix (ECM) was bioactive and could promote tissue repair in vivo.

However, there is a need to control the stability, viability and risk of uncontrolled action and differentiation of implanted stem cells. The logistics of transporting the stem cell sheet from a GMP facility to an operation theatre also requires careful planning to maintain the biological activity of the stem cells. The transplantation of stem cells for tissue regeneration is in its infancy due to the safety concerns.

Therefore, there is a need for novel compositions and methods for musculoskeletal and connective tissue repair that enhance standardization, affordability, and clinical translatability of stem cell-based therapies.

### BRIEF SUMMARY OF THE INVENTION

The subject invention pertains to decellularized stem cell sheet and compositions thereof with retained biological activity. The present invention further relates to the methods of producing a decellularized stem cell sheet and methods of using the decellularized stem cell sheet for the promotion of tissue repair.

In certain embodiments, the decellularized stem cell sheet can retain the ultrastructural cues and biological activity of pretreated tendon-derived stem cell (TDSC) sheet. In certain embodiments, the decellularized stem cell sheet, including a decellularized TDSC (dTDSC) sheet can promote tendon graft healing after anterior cruciate ligament reconstruction. In certain embodiments, the decellularized stem cell sheet, including the dTDSC sheet, can express growth factors, such as, for example, bone morphogenetic protein-2 (BMP-2) and vascular endothelial growth factor (VEGF). In certain embodiments, the extracellular matrix (ECM) of the decellularized stem cell sheet, containing both collagenous and non-collagenous proteins, can provide both ultrastructural and biochemical cues to promote cell differentiation as well as biochemically and physically protect the wrapped graft tissue from inflammatory cytokine- and matrix metalloproteinase-induced tissue damage during healing. In certain embodiments, the use of the decellularized stem cell sheet, including the dTDSC sheet, for tissue repair can eliminate the use of synthetic scaffold, support homogeneous delivery of healing-promoting factors, is arthroscopy-compatible, can be tailor-made to different sizes and shapes, and can be prepared from cells isolated from surgical waste.

In certain embodiments, the decellularized stem cell sheet, including the dTDSC sheet, can have lower immunogenicity when compared to a cellular stem cell sheet due to cell removal. In certain embodiments, the decellularized stem cell sheet, does not have issues pertaining to cell stability, viability, and/or risk of uncontrolled action and differentiation of implanted cells. In certain embodiments, the reproducibility of the manufacturing process of the decellularized stem cell sheet can be higher than a cellular stem cell sheet and transportation to operation theatres can be easier.

In certain embodiments, the decellularized stem cell sheet can be used as a bioactive material for the promotion of tissue repairs and/or as a scaffold for the synthesis of bio-artificial tissue. In certain embodiments, the tissues to which the decellularized stem cell sheet can be applied include, for example, tendons and ligaments, cartilage, bone, muscle, or skin. In certain embodiments, the decellularized stem cell sheet can be used for tendon-bone junction repair, tendon window defect, tendon and ligament rupture, cartilage repair, bone repair, muscle repair, and skin repair. In certain embodiments, the decellularized stem cell sheet can be used as a scaffold for other cell types or growth factors for the promotion of tissue repair or for the synthesis of bio-artificial tendon and ligament graft tissue for tendon/ligament replacement.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1H. Procedures for applying the decellularized tendon-derived stem cell (dTDSC) sheet to the graft-to-bone tunnel interface during ACLR. (FIGS. 1A-1B) Harvest the ipsilateral flexor digitorum longus tendon as the tendon graft. (FIGS. 1C-1D) Create the tibial and femoral bone tunnels. (FIGS. 1E-1F) Wrap the dTDSC sheet to the graft. (FIG. 1G) Insert the tendon graft into bone tunnels. (FIG. 1H) Fix the tendon graft with suture tied over the neighboring periosteum and close the soft tissues in layers.

FIGS. 2A-2H. Characterization of TDSCs. Bright-field image of TDSCs cultured for (FIG. 2A) 1 day; (FIG. 2B) 3 days; and (FIG. 2C) 7 days; Scale bars: 100 µm. (FIG. 2D) Side scatter versus forward scatter plot showing the TDSCs. (FIG. 2E) Histogram showing the absence of expression of CD31 in the isolated TDSCs. (FIG. 2F) Histogram showing the expression of CD90 in the isolated TDSCs. (FIG. 2G) Histogram showing the expression of CD44 in the isolated TDSCs (blue peaks). (FIG. 2H) Photomicrographs showing osteogenic, chondrogenic and adipogenic differentiation of TDSCs after in vitro induction and evaluated by Alizarin red S staining, alcian blue staining and Oil red O staining, respectively.

FIGS. 3A-3C Optimization of decellularization protocol of TDSC sheet. (FIG. 3A) Effect of DNase I on the decellularization efficiency of Triton X-100. TDSC sheets were treated with 0.1% Triton X-100 for 2 h, followed by treatment with DNase I (100 U/mL) for various times. The decellularization efficiency was examined by DAPI staining. (i) TDSC sheet control; (ii) DNase 1 h; (iii) DNase 2 h; (iv) DNase 3 h; (v) DNase 4 h; (vi) DNase 5 h. Scale bar: 50 µm. Treatment with TDSC sheets with 0.1% Triton X-100, followed by 100 U/mL DNase I treatment for 5 h effectively removed cells (FIG. 3B) Effect of higher concentration of Triton X-100 (0.3%) for a shorter time (30 min) in 10 mM Tris + 25 mM EDTA buffer, followed by treatment with DNase I (150 U/mL) for various times on the decellularization efficiency. The decellularization efficiency was examined by H&E staining (i-iv), DAPI staining (v-viii) and dsDNA content (ix). (i, v) TDSC sheet control; (ii, vi) DNase 1 h; (iii, vii) DNase 1.5 h; (iv, viii) DNase 2 h; scale bar: 50 µm. (ix) Bar chart showing the dsDNA content of TDSC sheet control and after decellularization & DNase treatment for various times. Treatment of TDSC sheets with higher concentration of Triton X-100 in Tris-EDTA buffer for a shorter time, followed by DNase I treatment for 2 h effectively removed the cells, maintained the ECM structure and removed the dsDNA. However, the bioactive factors could not be detected in the dTDSC sheets. (FIG. 3C) Effect of aprotinin in preserving the biological factors in the TDSC sheet decellularized by Triton X-100, Tris-EDTA and DNase I. Aprotinin (1 µg/mL) was added to the Tris-EDTA buffer for decellularization. BMP-2 and VEGF could be detected in the dTDSC sheets as shown by Western blotting.

FIGS. 4A-4N. Characterization of dTDSC sheet as compared to the TDSC sheet. (FIGS. 4A-4B) Gross morphology, scale bar: 1 cm; (FIGS. 4C-4D) H&E staining, scale bar: 100 µm; (FIGS. 4E-4F) DAPI staining, scale bar: 100 µm; (FIGS. 4G-4H) SEM analysis, scale bar: 50 µm; (FIG. 4I) DNA agarose gel (1%) showing size of DNA fragments; (FIG. 4J) bar chart showing the dsDNA content; (FIG. 4K) bar chart showing the collagenous protein content; (FIG. 4L) bar chart showing the non-collagenous protein content; (FIGS. 4M-4N) bar charts showing the expression of BMP-2 and VEGF as assessed by ELISA. **p < 0.01

FIGS. 5A-5G. Mineralized tissue formation inside bone tunnel after ACLR. Representative micro-CT images showing the newly formed mineralized tissue inside different tunnel regions in the control and dTDSC sheet groups at week 2 and week 6 post-ACLR (FIG. 5A); Bar charts showing (FIGS. 5B, 5D, 5F) bone mineral density (BMD) (mg HA/cm³) and (FIGS. 5C, 5E, 5G) bone volume/total volume (BV/TV) of newly formed mineralized tissue inside the (FIGS. 5B-5C) femoral tunnel, (FIGS. 5D-5E) epiphyseal region, and (FIGS. 5F-5G) metaphyseal region of the tibial tunnel in the control and dTDSC sheet groups at week 2 and week 6 post-ACLR. *p < 0.05, **p < 0.01

FIGS. 6A-6C. Photomicrographs showing graft healing. H&E staining at the (FIG. 6A) femoral tunnel and (FIG. 6B) the intra-articular graft mid-substance in the control group and dTDSC sheet group at week 2 and week 6 post-ACLR. The right panel showed the polarized microscopic images of the same view; (FIG. 6C) Safranin-O staining at the femoral and tibial tunnels. Yellow arrows: blood vessels; black arrowheads: chondrocyte-like cells; white arrows: Sharpey’s fibers; B: bone; I: interface; T: tendon; Scale bar =500 µm

FIGS. 7A-7D. Biomechanical properties of the bone-graft-bone complex after ACLR. Bar charts showing the (FIG. 7A) ultimate failure load and (FIG. 7B) stiffness of the bone-graft-bone complex at week 2 and week 6 after ACLR in the control group and dTDSC sheet group. (FIGS. 7C-7D) Representative force-displacement curves of the bone-graft-bone complex in different groups at week 2 and week 6 after ACLR during the pull-out test. *p < 0.05, **p < 0.01, ***p < 0.001.

FIGS. 8A-8D. Immunohistochemical staining of iNOS and CD206 at the femoral tunnel interface at week 2 and week 6 after ACLR. (FIG. 8A) Photomicrographs showing the immunohistochemical staining of iNOS at the femoral tunnel interface, scale bar: 100 µm; (FIG. 8B) Bar chart showing the percentage of iNOS⁺ cells at the femoral tunnel interface; (FIG. 8C) Photomicrographs showing the immunohistochemical staining of CD206 at the femoral tunnel interface, scale bar: 100 µm; (FIG. 8D) Bar chart showing the percentage of CD206⁺ cells at the femoral tunnel interface. Black arrows: positive stained cells; B: bone; I: interface; T: tendon; **p < 0.01

FIGS. 9A-9F. Immunohistochemical staining of MMP-1, MMP-13 and TIMP-1 at the femoral tunnel interface at week 2 and week 6 after ACLR. (FIG. 9A) Photomicrographs showing the immunohistochemical staining of MMP-1 at the femoral tunnel interface, scale bar: 100 µm; (FIG. 9B) Bar chart showing the signal intensity score of MMP-1 at the femoral tunnel interface; (FIG. 9C) Photomicrographs showing the immunohistochemical staining of MMP-13 at the femoral tunnel interface, scale bar: 100 µm; (FIG. 9D) Bar chart showing the signal intensity score of MMP-13 at the femoral tunnel interface; (FIG. 9E) Photomicrographs showing the immunohistochemical staining of TIMP-1 at the femoral tunnel interface, scale bar: 100 µm; (FIG. 9F) Bar chart showing the signal intensity score of TIMP-1 at the femoral tunnel interface. B: bone; I: interface; T: tendon; **p < 0.01

FIGS. 10A-10B. Photomicrographs showing graft healing at the tibial tunnel after ACLR. The left panel of each figure showed the H&E staining while the right panel showed the polarized microscopic images of the same view.(FIG. 10A) epiphyseal region of tibial tunnel; (FIG. 10B) metaphyseal region of tibial tunnel in the control group and dTDSC sheet group at week 2 and week 6 post-ACLR. Yellow arrows: blood vessels; black arrowheads: chondrocyte-like cells; white arrows: Sharpey’s fibers; B: bone; I: interface; T: tendon; scale bar =500 µm

FIGS. 11A-11C. Immunohistochemical staining of iNOS at the tibial graft-to-bone tunnel interface and the graft mid-substance at week 2 and week 6 after ACLR. (FIG. 11A) Photomicrographs showing the immunohistochemical staining of iNOS at the tibial graft-to-bone tunnel interface and the graft mid-substance in the control group and dTDSC sheet group, scale bar: 100 µm. Bar charts showing the percentage of iNOS⁺ cell at the (FIG. 11B) tibial tunnel interface; and (FIG. 11C) graft mid-substance. Black arrows: iNOS⁺ cells; B: bone; I: interface; T: tendon; *p < 0.05, **p < 0.01

FIGS. 12A-12C. Immunohistochemical staining of CD206 at the tibial graft-to-bone tunnel interface and the graft mid-substance at week 2 and week 6 after ACLR. (FIG. 12A) Photomicrographs showing the immunohistochemical staining of CD206 at the tibial graft-to-bone tunnel interface and the graft mid-substance in the control group and dTDSC sheet group, scale bar: 100 µm. Bar charts showing the percentage of CD206⁺ cell at the (FIG. 12B) tibial tunnel interface; and (FIG. 12C) graft mid-substance. Black arrows: CD206⁺ cells; B: bone; I: interface; T: tendon; **p < 0.01.

FIGS. 13A-13C. Immunohistochemical staining of MMP-1 at the tibial graft-to-bone tunnel interface and the graft mid-substance at week 2 and week 6 after ACLR. (FIG. 13A) Photomicrographs showing the immunohistochemical staining of MMP-1 at the tibial graft-to-bone tunnel interface and the graft mid-substance in the control group and dTDSC sheet group, scale bar: 100 µm. Bar charts showing the signal intensity score of MMP-1 expression at the (FIG. 13B) tibial tunnel interface; and (FIG. 13C) graft mid-substance. B: bone; I: interface; T: tendon; **p < 0.01.

FIGS. 14A-14C. Immunohistochemical staining of MMP-13 at the tibial graft-to-bone tunnel interface and the graft mid-substance at week 2 and week 6 after ACLR. (FIG. 14A) Photomicrographs showing the immunohistochemical staining of MMP-13 at the tibial graft-to-bone tunnel interface and the graft mid-substance in the control group and dTDSC sheet group, scale bar: 100 µm. Bar charts showing the signal intensity score of MMP-13 expression at the (FIG. 14B) tibial tunnel interface; and (FIG. 14C) graft mid-substance. B: bone; I: interface; T: tendon; **p < 0.01.

FIGS. 15A-15C. Immunohistochemical staining of TIMP-1 at the graft-to-bone tunnel interface and the graft mid-substance at week 2 and week 6 after ACLR. (FIG. 15A) Photomicrographs showing the immunohistochemical staining of TIMP-1 at the tibial graft-to-bone tunnel interface and the graft mid-substance in the control group and dTDSC sheet group, scale bar: 100 µm. Bar charts showing the signal intensity score of TIMP-1 expression at the (FIG. 15B) tibial tunnel interface; and (FIG. 15C) graft mid-substance. B: bone; I: interface; T: tendon. *p < 0.05, **p < 0.01.

FIG. 16. Immunohistochemical staining of MMP-1, MMP-13, TIMP-1 of the contralateral intact ACL at week 2 and week 6 after ACLR. scale bar: 100 µm

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X ± 10%). In other contexts the term “about” is used provides a variation (error range) of 0-10% around a given value (X ± 10%). As is apparent, this variation represents a range that is up to 10% above or below a given value, for example, X ± 1%, X ± 2%, X ± 3%, X ± 4%, X ± 5%, X ± 6%, X ± 7%, X ± 8%, X ± 9%, or X ± 10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “subject” refers to an animal, needing or desiring delivery of the benefits provided by a therapeutic compound. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. These benefits can include, but are not limited to, the treatment of a health condition, disease or disorder; prevention of a health condition, disease or disorder; immune health; enhancement of the function of an organ, tissue, or system in the body. The preferred subject in the context of this invention is a human. The subject can be of any age or stage of development, including infant, toddler, adolescent, teenager, adult, or senior.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. The term nucleic acid is used interchangeably with gene, cDNA, and mRNA encoded by a gene.

As used herein, the term “treatment” refers to eradicating, reducing, ameliorating, or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, the term “decellularization,” refers to the substantial (i.e., at least 75%, 80%, 85%, 90%, 95%, 96%, 97%, 98%, 99%, or greater) removal of cellular components by the use of chemical and biological means. Decellularization is considered to be successful if (1) less than 50 ng dsDNA per mg extracellular matrix (ECM) dry weight persists or more than 90% of dsDNA content is removed; (2) no DNA fragments remaining that are greater than 200 base pairs in length; and/or (3) there is no visible nuclear material in tissue sections stained in H&E and DAPI^{10,17}.

As used herein, the “stem cell” refers to a cell exhibiting self-renewal and multi-lineage differentiation potential.

As used herein, the term “cellular components” refers to cell membranes, cytoplasm, dsDNA, and organelles (e.g., nucleus, mitochondria, endoplasmic reticulum, Golgi apparatus, lysosome) that make up a cell.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims.

### Decellularized Stem Cell Sheets and Methods of Decellularizing the Cell Sheets

In certain embodiments, a decellularized cell sheet is formed using a stem cell for the promotion of tissue repair and/or can be used as a scaffold for the synthesis of bio-artificial tissue.

In certain embodiments, the synthesis of a decellularized stem cell sheet involves isolating stem cells and then preparing a stem cell sheet and decellularizing the cell sheets afterwards.

In certain embodiments, the stem cell used in the invention is an adult stem cell. The stem cell is isolated from animal or human tissues. The stem cell used for the production of the cell sheet can be autologous or allogeneous. The stem cell can be isolated from, for example, a tendon and ligament, bone marrow, adipose tissue, umbilical cord blood, or dental pulp. In an embodiment, stem cell that proliferates fast, shows high colony-forming ability, and exhibits high expression of multi-lineage differentiation markers and produces high level of ECM has the advantage of forming the cell sheet as a result of shortening the in vitro cell culture time and increasing the success of forming the cell sheet in vitro upon treatment. In preferred embodiments, the stem cell is tendon-derived stem cell (TDSC). TDSC is stem cell isolated from tendon.^{64,33}

In certain embodiments, stem cells can be isolated from tissues, such as, for example, TDSC from tendon or bone marrow-derived stromal cells (BMSC) from bone marrow according to established protocols.^{33,64} In certain embodiments, the stem cells can be plated at about 50 cells/cm² to about 2000 cells/cm², about 100 cells/cm² to about 1000 cells/cm², or about 500 cells/cm² in a 100-mm dish and cultured in low-glucose Dulbecco’s modified Eagle’s medium, 10% fetal bovine serum (FBS), 50 µg/mL penicillin, 50 µg/mL streptomycin, and 100 µg/mL neomycin (complete culture medium) or in alpha minimum essential medium (α-MEM; Gibco), 10% FBS, 100 U/mL of penicillin, 100 µg/mL of streptomycin, and 2 mM L-glutamine until the cells reached confluence. In certain embodiments the stem cells are adult stem cells. Then the stem cells, including, for example, TDSC, can be treated with connecting tissue growth factor (CTGF), optionally, at a concentration of about 25 ng/mL, and collagen-forming bioactive factors, such as, for example, ascorbic acid, optionally, at a concentration of about 25 µmol/L, for about 2 weeks to about 4 weeks at about 25° C. to about 37° C. and about 5% CO₂^{37,38} and as described in U.S. Pat. No. 8,945,536, each of which are hereby incorporated by reference in its entirety, resulting in the formation of a cell sheet. The cell sheet can be detached from the culture dish by rinsing with phosphate-buffered saline (PBS) or NaCl, such as, for example, 0.9% NaCl.

In certain embodiments, a decellularization solution can be added to the detached cell sheet. In certain embodiments, the decellularization solution can comprise about 0.05% to about 0.5% Triton X-100, about 1 mM to about 20 mM Tris, about 10 to about 40 mM Ethylenediaminetetraacetic acid (EDTA), and about 0.5 µg/mL to about 3 µg/mL aprotinin or, in preferred embodiments, about 0.3% Triton X-100, about 10 mM Tris, about 25 mM EDTA, and about 1 µg/mL aprotinin, and can be incubated with the cell sheet for about 10 mins to about 120 mins, or about 30 mins at about 4° C. to about 37° C. In certain embodiments, the cell sheet can then be rinsed with PBS or NaCl, such, as for example, 0.9% NaCl, for about 1 min to about 24 h or about 12 h at about 4° C. to about 37° C. and treated with 150 U/mL DNase I for about 30 mins to about 180 mins or about 120 mins at about 4° C. to about 37° C. and then can be rinsed in PBS for about 1 h to about 36 h at about 4° C. to about 37° C. The resulting decellularized cell sheet can then be detached from the culture dish by rinsing with PBS or NaCl, such, as for example, 0.9% NaCl. The rinsing steps throughout the method of decellularizing the stem cell sheet can occur for about 1 min to about 36 hours, about 1 hour to about 24 hours, or about 12 hours to about 24 hours at a temperature of about 4° C. to about 37° C.

In certain embodiments, the decellularized cell sheet as disclosed herein can be prepared by a method comprising treating stem cells to remove cellular components. In certain embodiments, at least about 75%, about 80%, about 85%, about 90%, about 95%, about 96%, about 97%, about 98%, about 99%, or greater amount of double-strand nucleic acid material can be removed from the stem cell sheets.

In certain embodiments, the dTDSC sheet can contain collagenous and non-collagenous protein, bioactive factors including growth factors, such as, for example, bone morphogenetic protein-2 (BMP-2) and vascular endothelial growth factor (VEGF) and small RNA, has no observable cells as shown by histology (DAPI) and electron microscopy, and has about at least about 98% of the dsDNA removed.

In certain embodiments, the decellularized cell sheet comprises the ECM of the cell. In certain embodiments, the ECM of the cell can contain factors including growth factors, such as, for example, BMP-2 and VEGF, and small RNA that are retained by that ECM. In certain embodiments, the RNA molecules of the decellularized cell sheet can be less than 200 base pairs in length. Cells and double strand DNA in the cells are removed. The decellularized cell sheet retains most of the collagenous proteins of the original cell sheet. Some but not all non-collagenase proteins are retained due to cell removal, which is expected as cellular components contain mostly non-collagenous proteins.

In certain embodiments, the decellularized cell sheet expresses BMP-2 and VEGF. In certain embodiments, the growth factors retained in the decellularized cell sheet can promote tissue repair. In certain embodiments, BMP-2 can promote osteogenic differentiation of stem cells and enhance bone formation. In certain embodiments, VEGF can enhance angiogenesis which is required for tissue healing after injury.

In some embodiments, the therapeutically effective amount of a composition comprising a decellularized stem cell sheet can be administered through topical administration or by direct application of the decellularized cell sheet to the site in need of repair, such as, for example a tendon, ligament, muscle, bone, cartilage, or skin. In certain embodiments, the larger the decellularized cell sheet that is applied topically or by direct application, the more biomaterial and bioactive factors in the decellularized cell sheet. In certain embodiments, the decellularized stem cell sheet can cover the entire injured site, such as, for example, wrap the whole tendon graft, or fill a site or gap, such as, for example the gap of the patellar tendon window injury.

### Methods of Use

In certain embodiments, the decellularized stem cell sheet can be used for enhancing tissue repair, particularly musculoskeletal and/or connective tissue. In certain embodiments, the decellularized stem cell sheet can be used to promote tendon-bone junction regeneration, such as, for example, tendon graft to bone tunnel healing and graft remodeling in ACL reconstruction, rotator cuff repair, or patellar bone-patellar tendon repair. Accordingly, the decellularized stem cell sheet can be used in a method of promoting tendon-bone junction regeneration in ACL reconstruction comprising the steps of wrapping the tendon graft prior to insertion into the bone tunnel during surgical reconstruction. In another embodiment, the decellularized cell sheet can be used for rotator cuff repair by suturing the decellularized cell sheet to the interface between tendon and bone.

In one embodiment, the decellularized stem cell sheet can be used for the repair of a window injury in the patellar tendon. The window injury of the patellar tendon is caused by removal of the patellar bone-patellar tendon-bone graft in anterior cruciate ligament (ACL) reconstruction. In the embodiments of the invention, the decellularized cell sheet is used in a method for enhancing the repair of window injury in the patellar tendon comprising the steps of rolling and inserting the cell sheet in the window defect.

In certain embodiments, the decellularized cell sheet of the invention can be used to enhance suture repair of tendon (e.g. Achilles tendon, flexor tendon) and ligament (e.g. posterior cruciate ligament, PCL; ACL) by wrapping the decellularized cell sheet around the rupture site.

In certain embodiments, the decellularized cell sheet can be used for the repair of bone fracture, osteoarthritis, osteo-chondro defect, muscle tear, skin wound or burn.

In certain embodiments, the decellularized cell sheet can be used as a scaffold in combination with other cell types, such as, for example, bone marrow-derived stromal cell (BMSC), chondrocyte, or growth factors, such as, for example, transforming growth factor-1 (TGF-β1) and transforming growth factor-beta2 (TGF-β2) for the promotion of tissue repair.

In certain embodiments, the decellularized cell sheet can be used as a scaffold to form bio-artificial tissue for tissue replacement. For example, the decellularized cell sheet can be used for synthesis of bio-artificial tendon and ligament graft tissue for tendon/ligament replacement. In certain embodiments, the decellularized cell sheet can be layered on biomaterials such as, for example, polylactic acid (PLA), poly(lactic-co-glycolic acid) (PLGA). Cells such as TDSC, adipose tissue-derived stromal cells (ADSC), bone marrow-derived stromal cells (BMSC) or umbilical cord blood stromal cells can then be seeded on the decellularized cell sheet. The construct can then be rolled and mechanically loaded in vitro or in vivo to induce the formation of bio-artificial tissues, including, for example, tendon/ligament. In certain embodiments, the extracellular matrix (ECM) of the decellularized stem cell sheet can provide both ultrastructural and biochemical cues to promote cell differentiation as well as biochemically and physically protect the wrapped graft tissue from inflammatory cytokine- (e.g., tumor necrosis factor-α (TNF-α)) and matrix metalloproteinase- (MMP) (e.g., MMP1, MMP13) induced graft tissue degeneration during healing.

In certain embodiments, the decellularized stem cell sheet can have a lower immunogenicity when compared to a cellular stem cell sheet due to cell removal. In certain embodiments, the decellularized stem cell sheet can increase the amount of anti-inflammatory and regenerative M2 macrophages and reduce the amount of proinflammatory M1 macrophages at the injured site after transplantation in a subject. In certain embodiments, the decellularized stem cell sheet does not have issues pertaining to cell stability, viability, and/or risk of uncontrolled action and differentiation of implanted cells. In certain embodiments, the reproducibility of the manufacturing process of the decellularized stem cell sheet can be higher than that of a cellular stem cell sheet; and transportation to operation theatres can be easier.

### MATERIALS AND METHODS

### TDSC Isolation and Culture

TDSCs were isolated from tendons of subject. Care was taken that only the midsubstance of tendon tissue was used for TDSC isolation. The tendon tissue was minced, digested with type I collagenase to yield a single cell suspension, plated at an optimal low cell density for the isolation of stem cells and cultured to form colonies. The expression of stem cell markers, clonogenicity and multi-lineage differentiation potential of the isolated cells were determined according to our established protocols.^{64,33} Only TDSCs at early passages were used for the formation of decellularized cell sheet.

### Preparation of the TDSC Cell Sheet

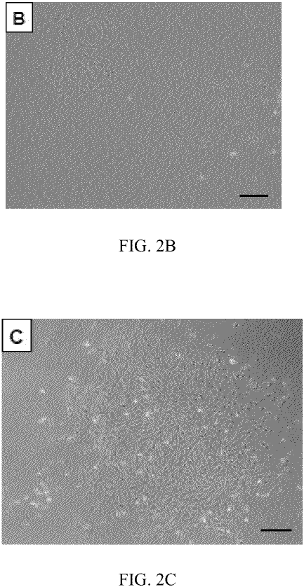

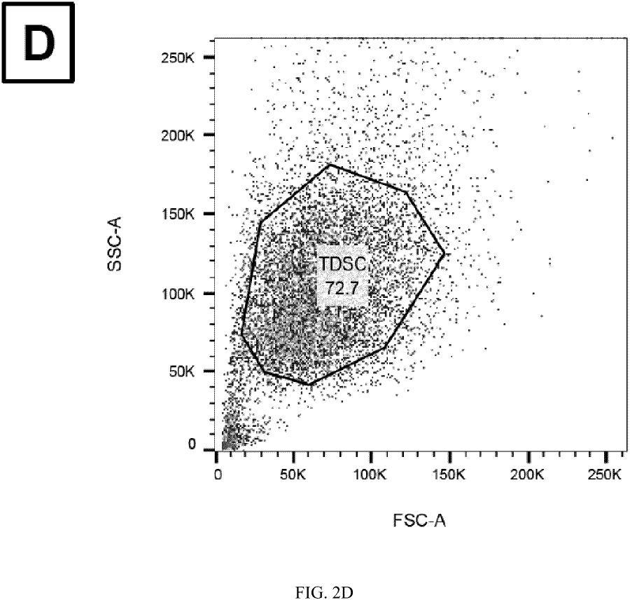

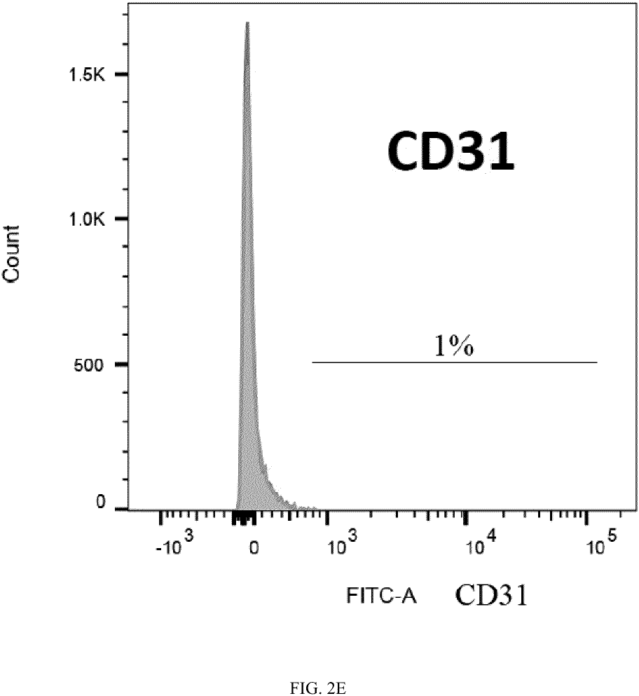

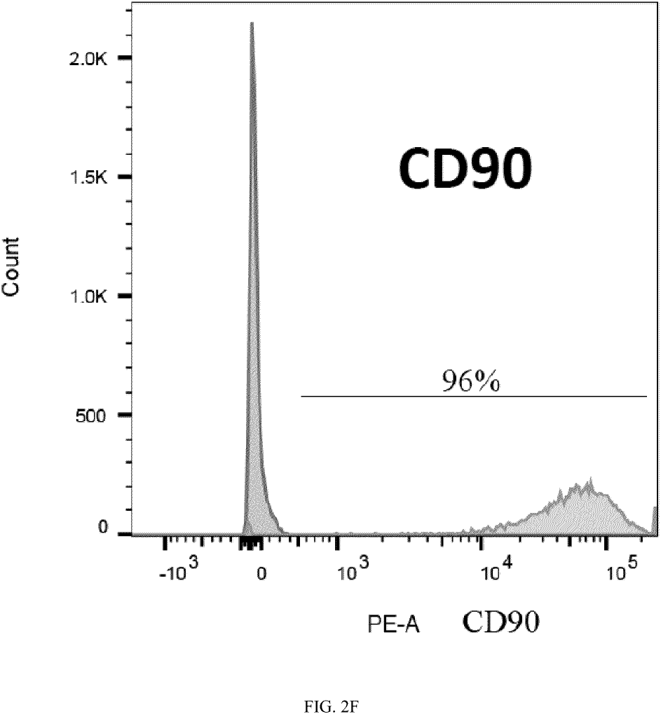

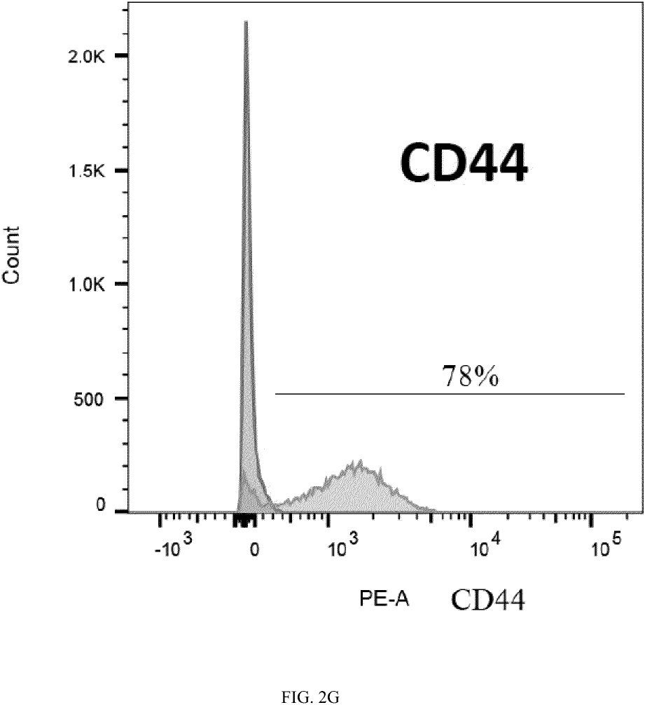

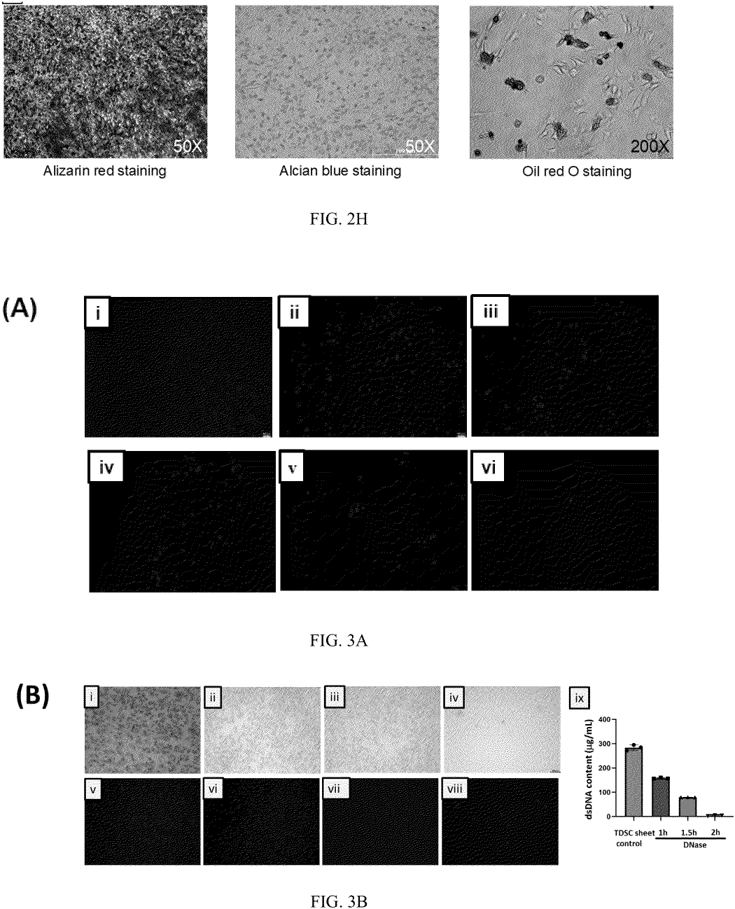

Achilles tendons from 4 male outbred SD rats (6 weeks old; weight, 200-220 g) were used for rat TDSC isolation. The TDSCs were isolated and characterized according to the published protocol³³ (FIGS. 2A-2D). The TDSCs expressed CD44 and CD90 but did not express CD31 (FIGS. 2E-2G). The TDSCs formed calcium nodules, expressed acid mucopolysaccharides and formed lipid droplets upon in vitro induction of osteogenic, chondrogenic and adipogenic differentiation (FIG. 2H). The formation of TDSC sheet was induced by treating TDSCs with CTGF and VitC in complete culture medium for 4 weeks as described previously³⁷.

### Decellularizing of the TDSC Sheet

Three decellularization approaches based on different mechanisms (freeze-thaw, Triton X-100, and SDS) were tested for the decellularization of TDSC sheets. The degree of decellularization was evaluated by hematoxylin & eosin (H&E) staining of ECM structure and 4′,6-diamidino-2-phenylindole (DAPI) staining of cell nuclei. Triton X-100 was subsequently selected for further evaluation, with modification of treatment dosage and time. DNase I, Tris-EDTA, and aprotinin were added in different experiments to find out the optimal conditions for complete decellularization while retaining the bioactive factors in the dTDSC sheets.

The final optimized decellularization protocol was as follows:

The TDSC sheets were placed in the decellularization solution, containing 0.3% Triton X-100 (Sigma-Aldrich Co., St. Louis, MO, USA), 25 mM EDTA (Sigma-Aldrich), 10 mM Tris (Sigma-Aldrich), and 1 µg/mL aprotinin (Roche, Mannheim, Germany) for 30 min, rinsed with PBS, treated with 150 U/mL DNase I (Roche) for 2 h, and rinsed in PBS for 24 h.

### In Vivo Effect of the dTDSC Sheet in ACLR

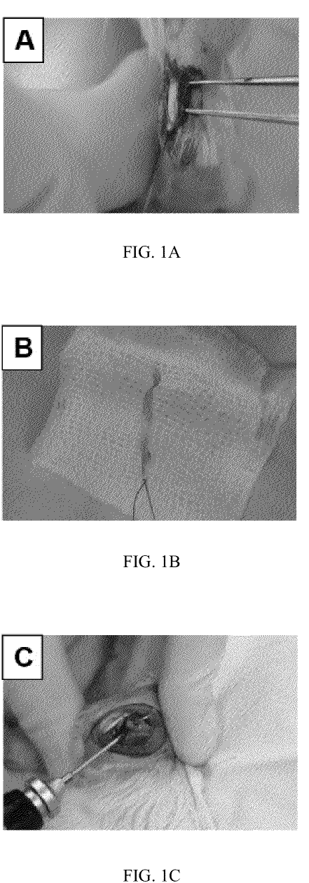

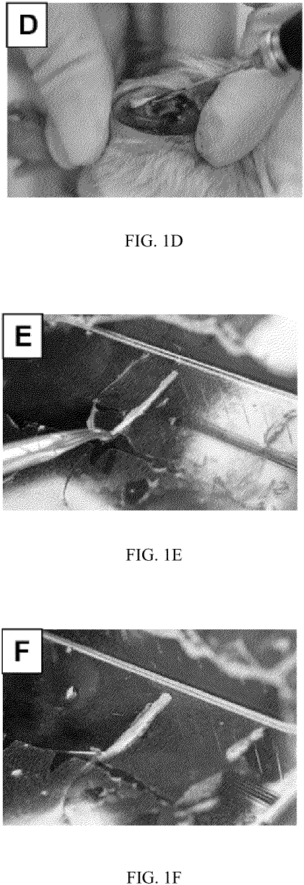

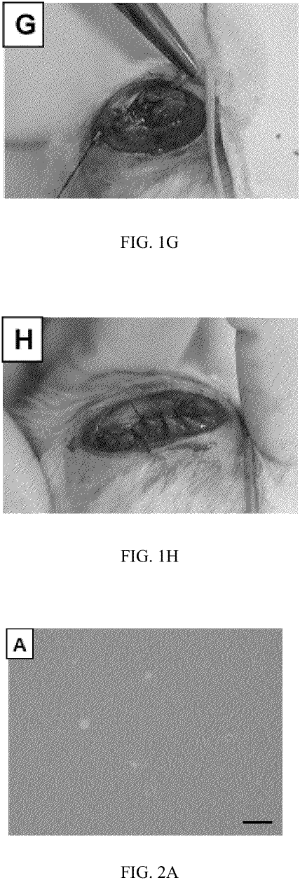

Unilateral ACLR was performed on the right knee according to our established protocol³⁷ (FIGS. 1A-1H). Sixty-four male SD rats (12 weeks old; weight, 350-380 g) were used. After anesthesia, the ipsilateral flexor digitorum longus tendon was harvested through a longitudinal medial incision (FIGS. 1A-1B). The ACL of the right knee was excised. Femoral (FIG. 1C) and tibial (FIG. 1D) tunnels of 1.1 mm in diameter and about 7 mm in length were created from the footprint of the original ACL to the anterolateral side of the femoral condyle and the medial side of the tibia, respectively, with an angle of about 55° to the articular surface. The rats were assigned either to the control group (n = 32) or dTDSC sheet group (n = 32). Rat dTDSC sheet (< 6 µm in thickness) was wrapped around the tendon graft (FIGS. 1E-1F) and routed through the bone tunnels (FIG. 1G) in the dTDSC sheet group. The graft was fixed at the femoral and tibial tunnel exits with the suture tied over the neighboring periosteum (FIG. 1H) and the soft tissues were closed in layers and verified by a Lachman test. The animals were allowed to have free cage movement immediately after operation. Except minor redness and swelling at the reconstructed site, no other postoperative complications were observed. At week 2 and week 6 post-ACLR, the knees were harvested for the measurement of mineralized tissue inside the bone tunnels by µCT (n = 6 per time point per group) followed by the assessment of the ultimate failure load and stiffness of the bone-graft-bone complex by biomechanical test. Another set of knees was harvested to meet the needs of the sample size of 10 per time point per group for the biomechanical test (n = 8 for the control group at week 6 due to sample damage during sample preparation). An additional set of knees was used to perform histological analysis of graft healing (n = 6 per time point per group). M1 macrophage marker iNOS and M2 macrophage marker CD206 as well as the expression of MMP-1, MMP-13, and TIMP-1 were examined by immunohistochemical staining (IHC) using the same samples (n = 6 per time point per group).

### CT Imaging and Image Analysis

A cone-beam CT system (µCT40, Scanco Medical AG, Bassersdorf, Switzerland) was used to assess the bone mass and density of newly formed mineralized tissue inside the bone tunnels according to the published protocol³⁶. Briefly, the region covering the entry and exit of the bone tunnel was scanned with vertical displacement of 30 mm for about 220 and 350 consecutive sections, respectively, for the femoral and tibial tunnels at a resolution of 35 µm. The sections were 3-dimensionally (3D) reconstructed and rotated to align the bone tunnel vertically using the built-in software. A circular region of interest (ROI) of 1.1 mm in diameter was used to represent the perimeter of the bone tunnel. Bone mineral density (BMD) and bone volume/total volume (BV/TV) were calculated for the volume of interest (VOI) covering the whole femoral tunnel, the epiphyseal, and metaphyseal tibial tunnels. About 50 sections were analyzed in each segment and were very consistent among different animals.

### Histology

Samples were fixed, decalcified, and embedded in paraffin as described previously³⁷. Mid-longitudinal sections of 7 µm thick and parallel to the direction of the bone tunnel were cut and stained with H&E and Safranin-O for the examination of graft healing under light microscopy (DM5500; Leica Microsystems Wetzlar GmbH, Wetzlar, Germany). The Sharpey’s fibers were examined under polarization using the same microscope. One section of the femoral tunnel, epiphyseal and metaphyseal regions of the tibial tunnel of each knee sample were assessed. Graft-to-bone tunnel healing was scored according to our established scoring system³⁴ (Table 1). A higher grade indicated a better outcome. The integrity of graft mid-substance was assessed by cellularity, vascularity, cell alignment, and collagen birefringence. Representative images were presented.

TABLE 1

The scoring system for the evaluation of graft healing in ACLR

| Histologic features | Score |
|---|---|
| Graft integrity | |
| 0% (of graft remnant) | 0 |
| 1% to ≤20% (of graft remnant) | 1 |
| 21% to ≤40% (of graft remnant) | 2 |
| 41% to ≤60% (of graft remnant) | 3 |
| 61% to ≤80% (of graft remnant) | 4 |
| 81% to ≤100% (of graft remnant) | 5 |
| Connection Between Tendon Graft and Bone | |
| 0% (of healing interface) | 0 |
| 1% to ≤20% (of healing interface) | 1 |
| 21% to ≤40% (of healing interface) | 2 |
| 41% to ≤60% (of healing interface) | 3 |
| 61% to ≤80% (of healing interface) | 4 |
| 81% to ≤100% (of healing interface) | 5 |
| Sharpey’s Fibers | |
| 0% (of healing interface) | 0 |
| 1% to ≤20% (of healing interface) | 1 |
| 21% to ≤40% (of healing interface) | 2 |
| 41% to ≤60% (of healing interface) | 3 |
| 61% to ≤80% (of healing interface) | 4 |
| 81% to ≤100% (of healing interface) | 5 |

### Biomechanical Testing

Biomechanical testing was done according to the previous studies^{36,37}. The whole joint was mounted onto a material testing machine (Hounsfield H25K-S; Hounsfield Test Equipment Ltd., Salfords, Redhill, United Kingdom) and loaded at a displacement rate of 20 mm/min until failure using a 50-N or 250-N load cell after 5 cycles of preconditioning at the maximum displacement of 0.5 mm. The ultimate failure load (the maximum load reached just before or at failure in the pull-out test) was recorded. The slopes of the linear portion of the force-displacement curve was calculated at 0.1 mm interval and the maximum slope was recorded as the stiffness of the sample. There was no big variation in the linear region used for the calculation of stiffness in different samples. Intact ACL of the contralateral limb of 6 animals were randomly selected from both groups at each time point as healthy controls.

### In Vitro Characterization of the dTDSC Sheet

The dTDSC sheet was characterized by (1) depletion of cells as assessed by H&E and DAPI staining (n=5-6/group); (2) DNA fragment length and dsDNA content as assessed by agarose gel and PicoGreen assay (P11496, Invitrogen, CA, USA), respectively (n=2-3/group); (3) collagenous and non-collagenous protein content as determined by Sirius Red F3BA staining and Fast Green FCF staining³², respectively (n=6/group); (4) surface topology as demonstrated by scanning electron microscopy (SEM) (n=3/group); and (5) expression of key growth factors including bone morphogenetic protein-2 (BMP-2) (ab213900, Abcam, Cambridge, Massachusetts, USA) and vascular endothelial growth factor (VEGF) (ab100786, Abcam) as determined by ELISA (n=4-5/group). Untreated TDSC sheets were used as the control for comparison.

The criteria for assessing successful decellularization were: (1) dsDNA content < 50 ng/mg ECM dry weight / > 90% of host DNA removed; (2) DNA fragment length < 200 bp; and (3) no visible cells in tissue sections stained in H&E and DAPI^{16,18}.

### Histological Analysis

The decellularization efficiency was evaluated by H&E and DAPI staining. Briefly, TDSC sheets and dTDSC sheets were smeared on slides, fixed for 2 h in 4% paraformaldehyde (PFA) solution, and stained with H&E. The stained slides were mounted with Prolong Gold mounting medium (Invitrogen, CA, USA) containing 0.2 µg/mL DAPI and observed under the microscope (Leica DM5500B, Leica Microsystems, Wetzlar, Germany) (n = 5 - 6 per group).

### DNA Fragment Length Assessment and dsDNA Quantification

Nucleic acid was extracted from TDSC sheets and dTDSC sheets using the phenol/chloroform method. The samples were digested with 0.1 mg/mL proteinase K (Thermo Scientific, IL, USA) for 2 h at 37° C., followed by centrifugation at 18,000 g for 10 min at room temperature. 9 µL of the nucleic acid and 1 µL of loading buffer (10×) were added to each sample, and then the mixtures were checked by electrophoresis using 1.0% agarose gel. The image was taken using the Gel Doc™ EZ imager (BIO-RAD, CA, USA). The dsDNA concentration was measured by the PicoGreen dsDNA assay kit (Invitrogen, CA, USA) (n = 2 per group for fragment length assessment and n = 3 per group for dsDNA quantification).

### Determination of Collagenous and Non-collagenous Proteins

The TDSC sheets and dTDSC sheets were plated in 6-well plate. For the assessment of collagenous proteins and non-collagenous proteins, the TDSC sheets and dTDSC sheets were stained with 0.1% Sirius Red F3BA and 0.1% Fast Green FCF, respectively for 30 min. The stained wells were observed under light microscopy (Leica DM5500B). The bound dyes were extracted by 0.1 M NaOH in absolute methanol (1:1, v:v) and the colour intensity was measured at OD 540 nm for Sirius Red F3BA and at OD 620 nm for Fast Green FCF (n = 6 per group).

### Surface Topology of TDSC Sheet Before and After Decellularization

The surface topology of TDSC sheets and dTDSC sheets was examined by SEM according to the standard protocol. Briefly, the samples were fixed in 2.5% glutaraldehyde in PBS for 4 h, washed, and then fixed in 1% osmium tetroxide, followed by dehydration in increasing graded ethyl alcohols. The samples were then vacuum-dried, sputter-coated with platinum, and then examined with a scanning electron microscope (QUANTA 400F, FEI, Tokyo, Japan). The TDSC sheets and dTDSC sheets were viewed at the magnification of ×500, ×2,000, and ×7,000 (n = 3 per group).

### Expression of Bioactive Factors

Soluble proteins were extracted from the TDSC sheets and dTDSC sheets with RIPA Lysis and Extraction Buffer (Thermo Scientific, IL, USA). Then the samples were homogenized. The extracted lysate was centrifuged at 15,000 RPM for 10 min, and the supernatant was collected. The concentration of total soluble protein was measured by bicinchoninic acid (BCA) protein assay (Thermo Scientific, IL, USA). The levels of BMP-2 and VEGF in the extracted lysate of each group were measured by rat BMP-2 ELISA Kit (ab213900, Abcam, Cambridge, Massachusetts, USA) and rat VEGF ELISA Kit (ab100786, Abcam, Cambridge, Massachusetts), respectively, according to the manufacturer’s instructions (n = 4 - 5 per group).

### Immunochemical Staining

IHC of inducible nitric oxide synthase (iNOS), cluster of differentiation 206 (CD206), matrix metalloproteinase (MMP)-1, MMP-13, and tissue inhibitor of metalloprotease (TIMP)-1 was done according to the protocol as described previously³⁵. Briefly, after rehydration, antigen retrieval, and blocking, the sections were stained with primary antibodies (polyclonal rabbit antibody against mouse/rat iNOS (ab15323; 1:100; Abcam, USA), polyclonal rabbit antibody against rat/human/mouse CD206 (ab64693; 1:100; Abcam, USA), polyclonal rabbit antibody against human/mouse/rat MMP-1(10371-1-AP; 1:100; Proteintech, USA), polyclonal rabbit antibody against human/mouse/rat MMP-13 (ab39012; 1:100; Abcam), polyclonal rabbit antibody against human/rat TIMP-1(ab61224; 1:100; Abcam)) at 4° C. overnight. The primary antibodies were replaced with blocking solution in the controls. HRP-conjugated goat anti-rabbit secondary antibody (ab6721; 1:1000; Abcam) was used, and the binding was visualized using the DAB Substrate Kit according to the manufacturer’s protocol (Thermo Scientific, CA, USA). After counterstaining with hematoxylin, the sections were examined under light microscopy (DM5500; Leica). The number of iNOS⁺ and CD206⁺ cells per 200 cells at different tunnel regions and graft mid-substance were counted. For MMP-1, MMP-13 and TIMP-1, the expression was examined and compared based on the signal intensity score calculated with the open-source software ImageJ (version 1.53k), and the IHC profiler plug-in developed by Varghese et al.⁵⁴ Representative images were presented.

### Power Calculation and Data Analysis

Quantitative data were presented as mean ± standard deviation (SD) and shown in bar charts. Representative histological images were shown. The data between groups were compared using the Mann-Whitney U test. All the data analysis was done using SPSS (version 27.0; SPSS). p < 0.05 was regarded as statistically significant.

The ultimate failure load of the healing complex of the dTDSC sheet group was compared to that in the control group at week 6.The ultimate failure load of the control group at week 6 was 8.934 ± 2.447 N and the ultimate load of the intact rat ACL was 64.17N ± 1.617 N. Assuming that the ultimate failure load of the dTDSC sheet group could reach 25% of the strength of intact ACL (i.e., 16.04 N), which was considered clinically significant, a sample size of 10/group could have a 80% chance of detecting the difference at α=0.05 using the Mann-Whitney U test. The sample sizes for cell culture, histology, IHC and SEM were based on our experience with these techniques.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Decellularized TDSC Sheet

TDSCs proliferated rapidly and formed colonies after seeding in the plastic culture dish for 7 days (FIGS. 2A-2C). They expressed stem cell-specific surface markers (96% fibroblastic marker CD90 and 78% MSC marker CD44) and were negative for the endothelial cell marker CD31, thus verifying the absence of contaminating endothelial cells (FIGS. 2E-2G). Rat TDSCs were treated as described in U.S. Pat. No. 8,945,536 to form a cell sheet.

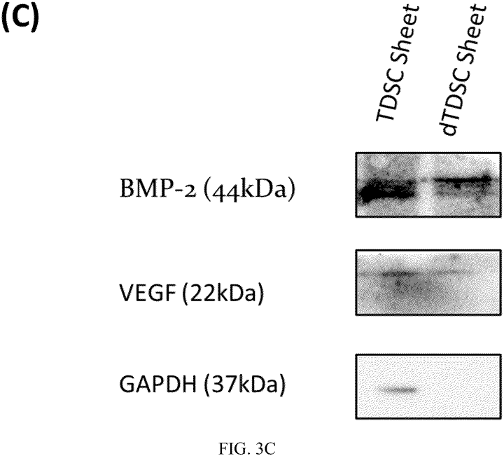

We tested the effects of freeze-thaw, Triton X-100 and SDS for the decellularization of TDSC sheets and the results with freeze-thaw and SDS were not satisfactory. Freeze-thawing of TDSC sheets twice at 30 min each was not effective in decellularization while freeze-thawing for 30 min followed by treatment with 0.5% SDS for 30 min successfully removed almost all cells as shown by DAPI staining but the ECM as shown by H&E staining was severely damaged. Treatment of TDSC sheets with DNase I for 5 hours after treatment with 0.1% Triton X-100 for 2 hours effectively removed the cells as shown by DAPI staining (FIG. 3A). However, the treatment time was too long. We hence increased the Triton X-100 concentration to 0.3% and shortened the treatment time to 30 min and added Tris-EDTA to aid the disruption of cell adhesion to ECM, prior to treatment of TDSC sheets with 150 U/mL DNase I for 2 h. This protocol could effectively remove the cells as shown by DAPI staining and dsDNA content (FIG. 3B). However, no active biological factors could be detected in the dTDSC sheets by Western blotting. We hence added 1 µg/mL aprotinin, which could preserve the active biological factors in the dTDSC sheets (FIG. 3C). An optimized decellularization protocol was hence established.

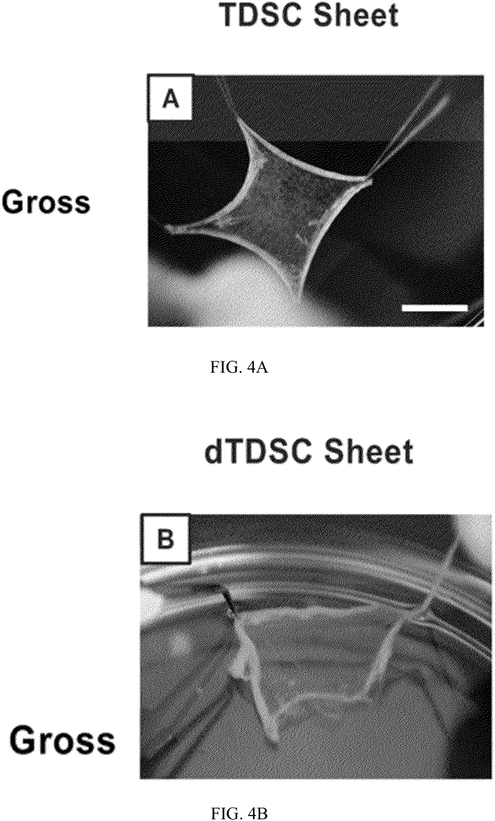

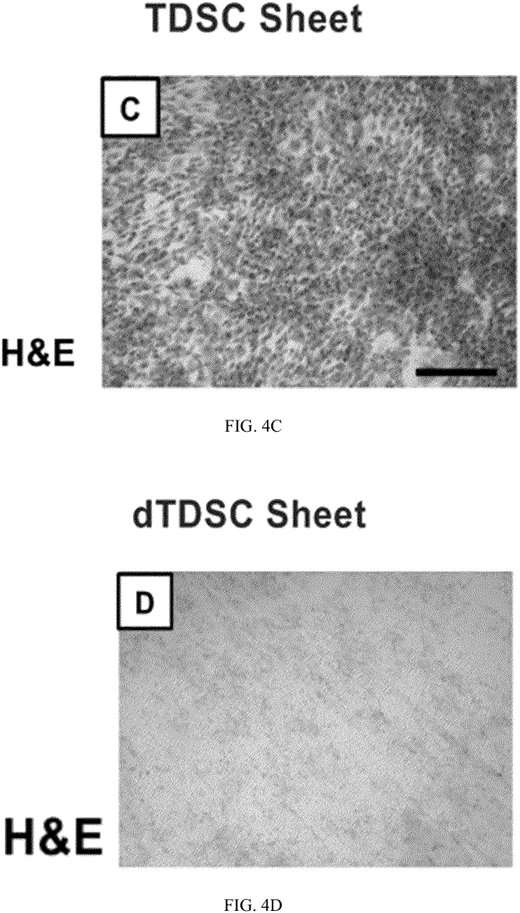

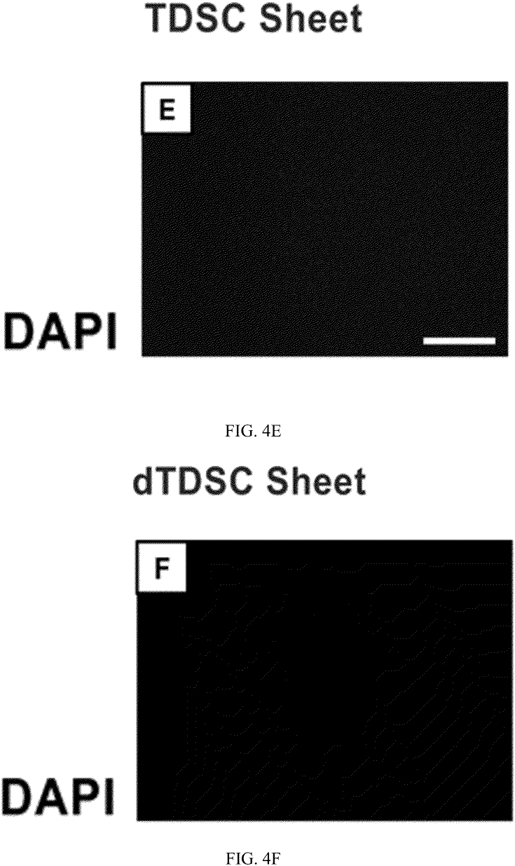

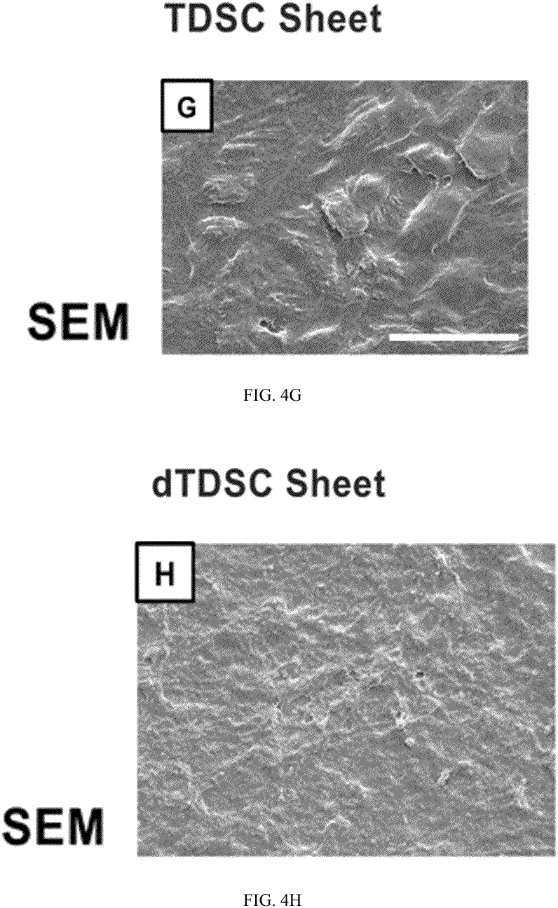

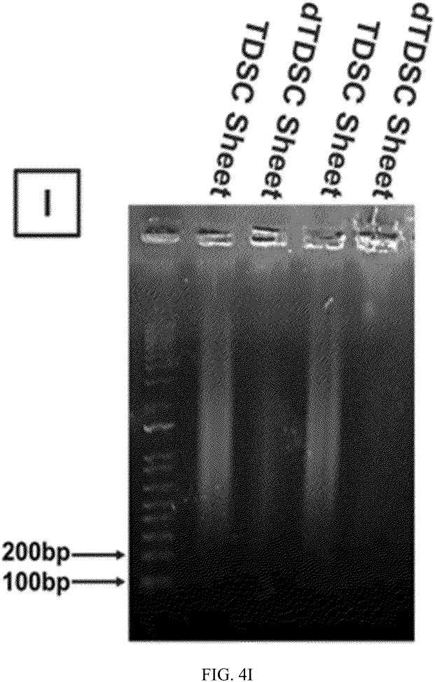

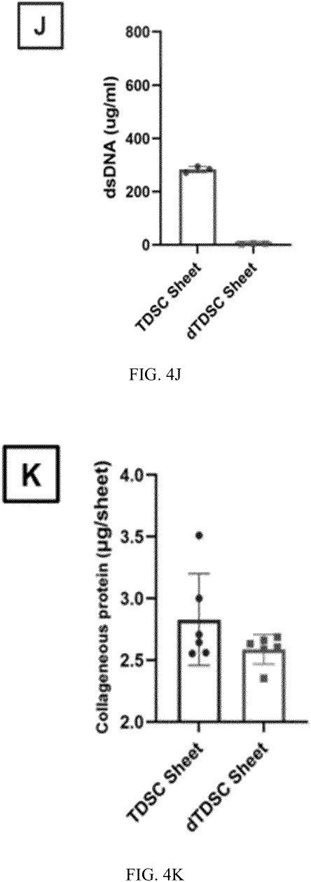

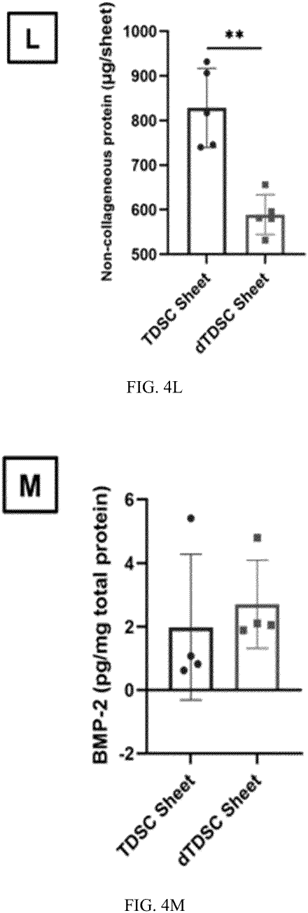

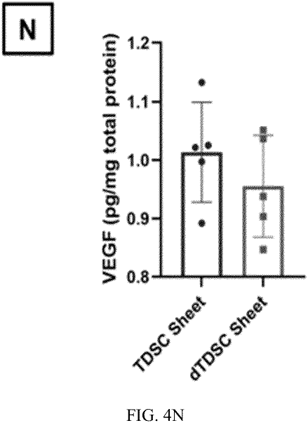

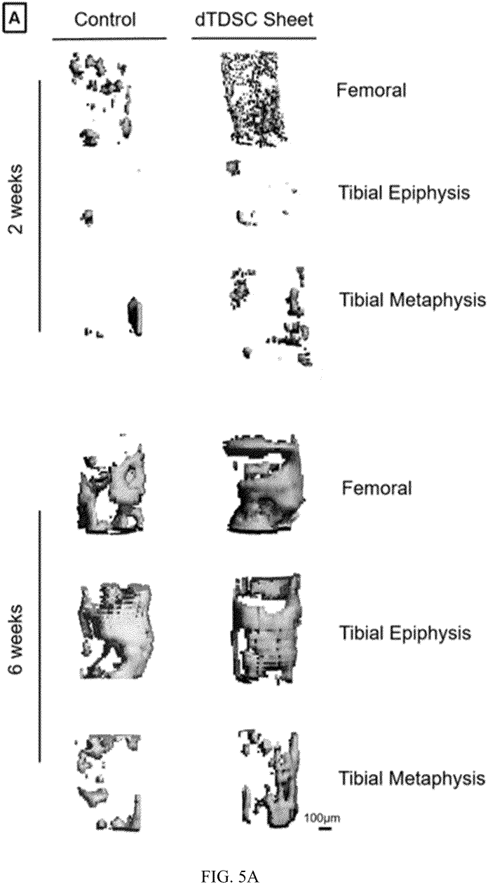

Different decellularization protocols using freeze-thaw, Triton X-100 and SDS were compared, and Triton X-100 best preserved the ECM structure and bioactive factors while eliminating the cells and genetic materials of the dTDSC sheet (data on file). After decellularization, the dTDSC sheet looked grossly similar to the TDSC sheet (FIGS. 4A-4B). The optimized TDSC sheet was successfully decellularized as shown by H&E staining, DAPI staining and scanning electron microscopy (SEM) (FIGS. 4C-4F, FIGS. 4G-4H). About 97.9% of collagenous proteins (FIG. 4K) and 71.2% of non-collagenous proteins (FIG. 4L) were retained in the dTDSC sheet compared to the TDSC sheet. The agarose gel electrophoresis showed that nucleic acid fragments of different sizes were present in the TDSC sheet, while no signal could be detected in the dTDSC sheet (FIG. 4I). The PicoGreen assay showed that about 98.4% of the nucleic acid was removed by the decellularization process (283.7 ± 11.72 µg/mL vs. 4.0 ± 1.81 µg/mL) (FIG. 4J). 97.9% of collagenous proteins were preserved in the dTDSC sheets and the difference was not statistically significant compared to the TDSC sheet (p>0.05) (FIG. 4K) while 71.2% of non-collagenous proteins were preserved (p=0.008) (FIG. 4L). The optimized decellularized protocol effectively preserved key growth factors including BMP-2 (FIG. 4M) and VEGF (FIG. 4N) in the dTDSC sheet. There was no significant difference in the amount of BMP-2 and VEGF in the TDSC sheet and dTDSC sheet (both p>0.05).

### Example 2—Healing Effects of the DTDSC Sheet

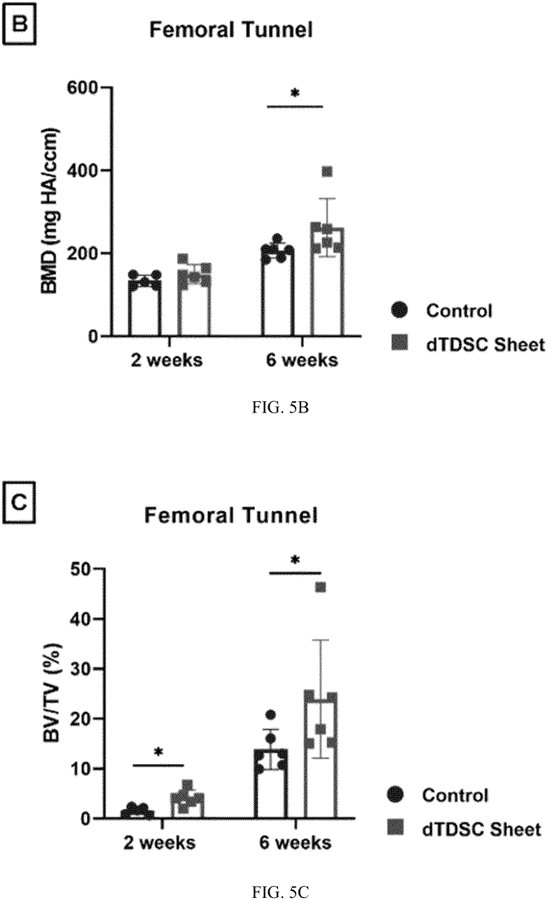

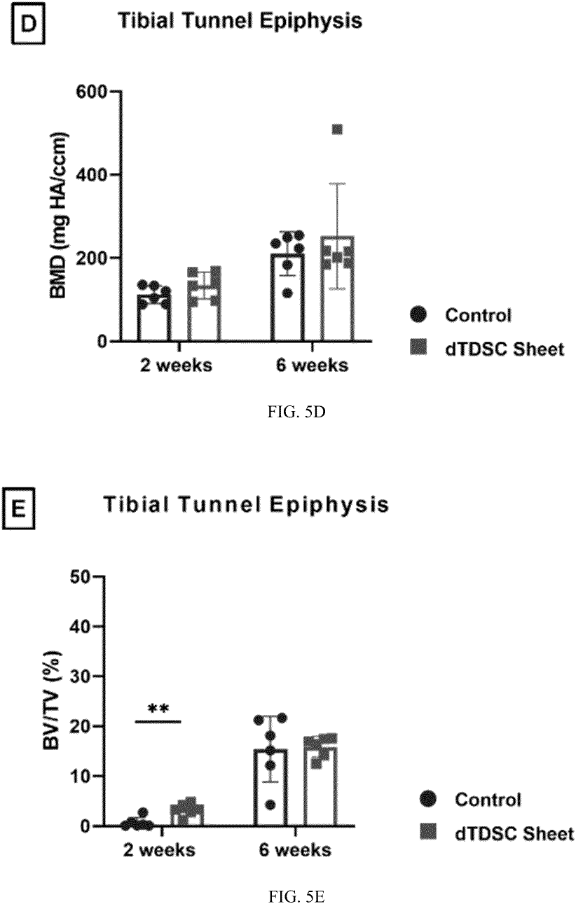

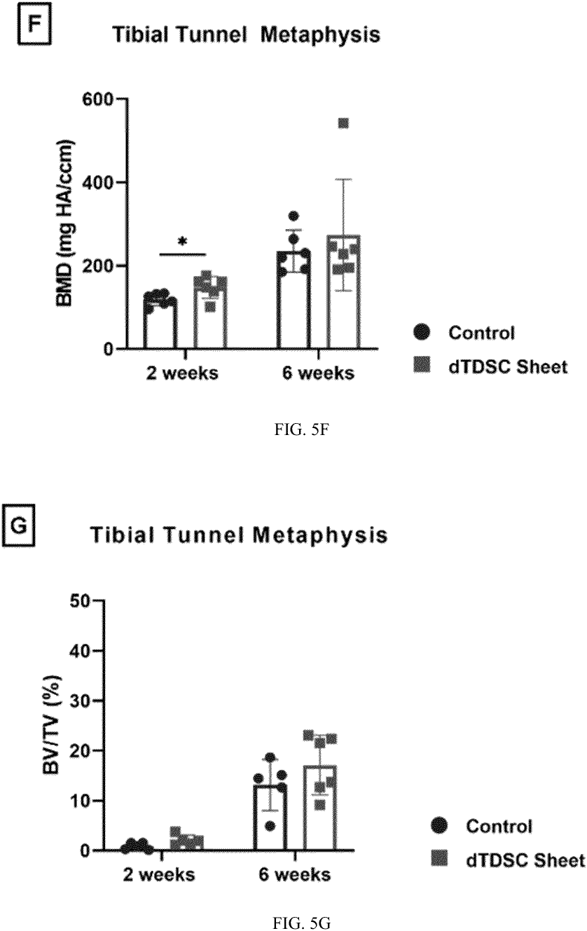

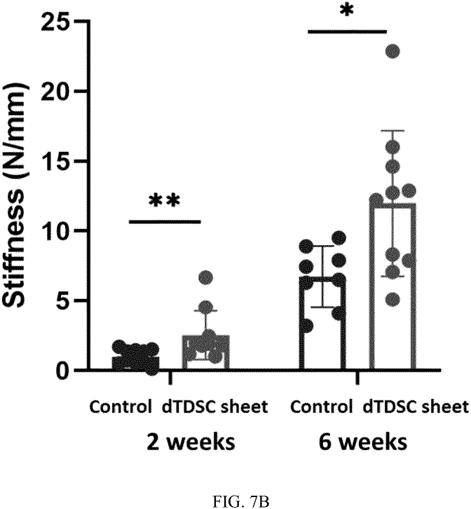

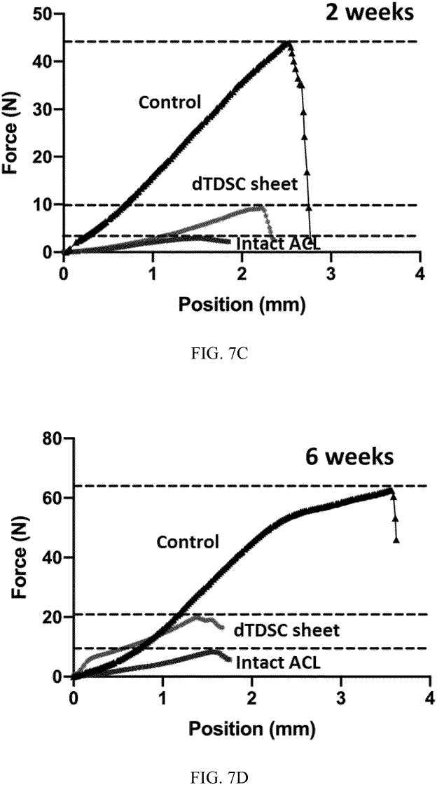

We wrapped the decellularized stem cell sheet around the tendon graft prior to its insertion into the bone tunnel in the animal model of anterior cruciate ligament reconstructive surgery. At week 2 after ACLR, there was significantly higher tunnel BMD at the tibial tunnel metaphysis and BV/TV at the femoral tunnel and tibial tunnel epiphysis in the dTDSC sheet group compared to those in the control group (FIGS. 5B-5G). At week 2 after ACLR, there was significantly higher BV/TV at the femoral tunnel (FIG. 5C) and at the epiphyseal region of tibial tunnel (FIG. 5E), as well as BMD at the metaphyseal region of tibial tunnel (FIG. 5F). At week 6 after ACLR, there was significantly higher BMD and higher BV/TV at the femoral tunnel in the dTDSC sheet group compared to those in the control group (FIGS. 5B-5C). The ultimate failure load and stiffness of the femur-graft-tibia complex were higher in the dTDSC sheet group compared with those in the untreated control group at week 2 and week 6 after ACLR (FIGS. 7A-7B).

### EXAMPLE 3—CT Imaging

CT image analysis showed significant higher tunnel BV/TV at the femoral tunnel at week 2 (165.9% increase, p=0.017), tunnel BMD (27.0% increase, p=0.015) and BV/TV (72.5% increase, p=0.041) at the femoral tunnel at week 6, tunnel BV/TV at the epiphyseal region of tibial tunnel at week 2 (363.8% increase, p=0.004) and tunnel BMD at the metaphyseal region of tibial tunnel at week 2 (25.0% increase, p=0.041) in the dTDSC sheet group compared to those in the control group (FIGS. 5A-5G).

### Example 4—Histology

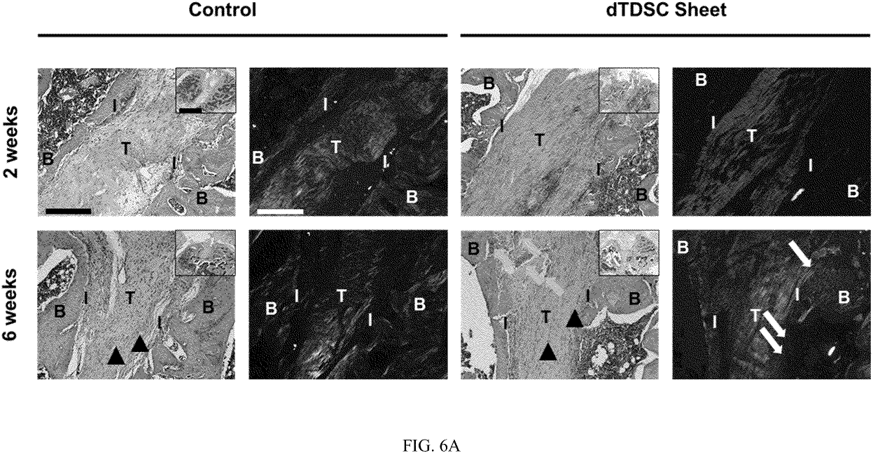

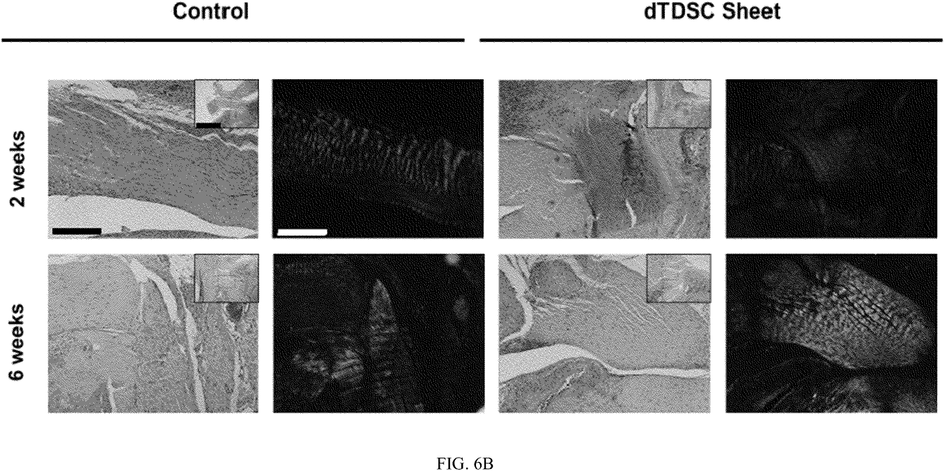

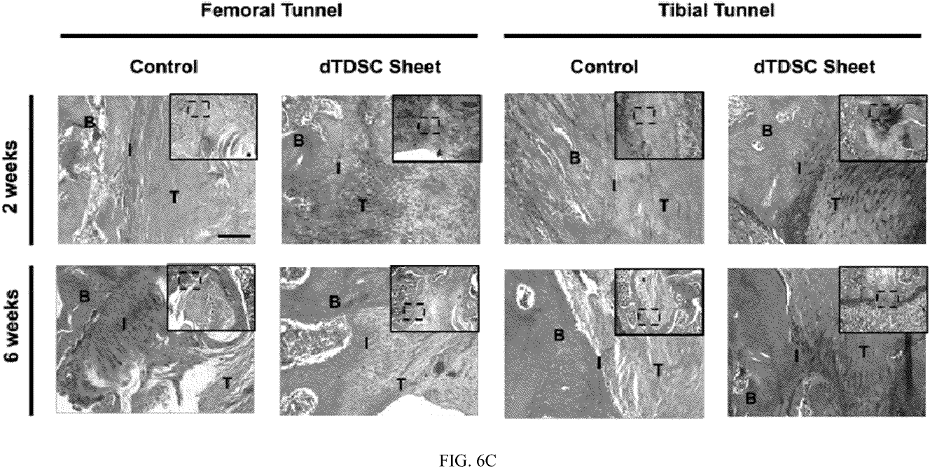

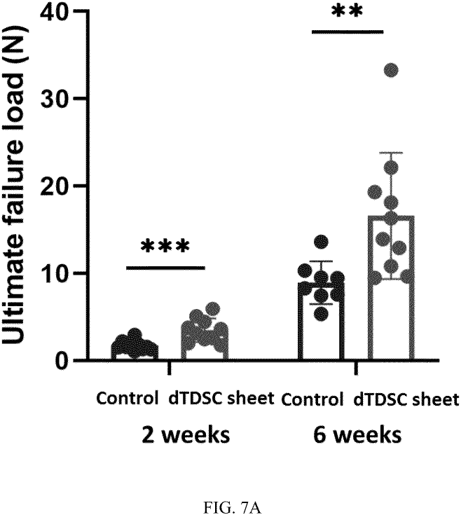

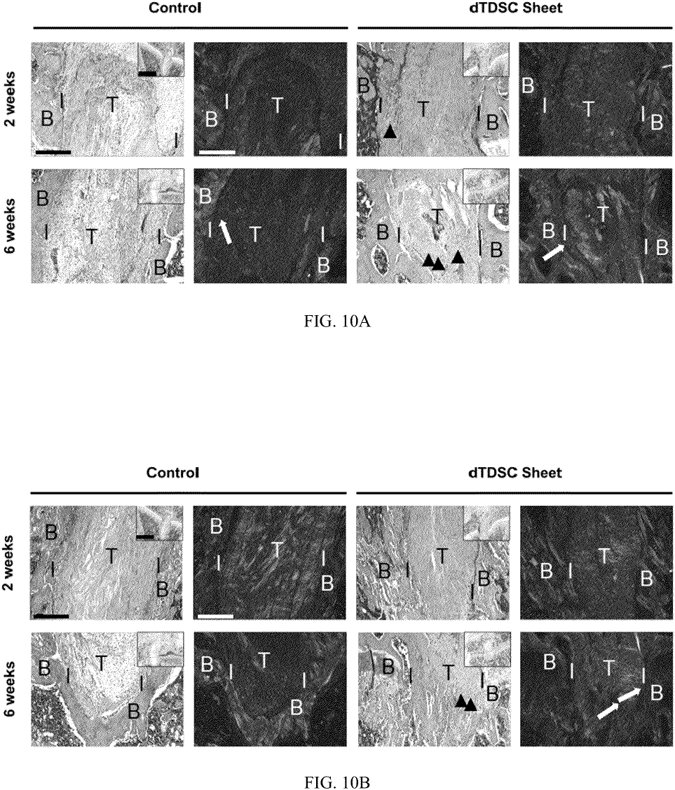

At the femoral tunnel, less fibrous tissue was observed at the interface in the dTDSC sheet group compared to that in the control group at week 2 (FIG. 6A; Table 2). Compared to week 2, there was less fibrous tissue and space at the tunnel interface in the control group, but the tunnel graft degenerated at week 6 (FIG. 6A). Meanwhile, better tendon graft integrity and more blood vessels (yellow arrow) were observed in the dTDSC sheet group at week 6. Chondrocyte-like cells (black arrowheads) were observed at the graft-bone tunnel interface or on the graft in both groups at week 6 (FIG. 6A). Histological scoring showed better tunnel graft healing in the dTDSC sheet group compared to that in the control group at week 6, due to higher tunnel graft integrity and slightly more Sharpey’s fibers (white arrows) at the tunnel interface (FIG. 6A; Table 2). Similar outcomes with better graft osteo-integration were detected in the tibial tunnel (FIGS. 10A-10B, Table 2). For the healing at the graft mid-substance, higher cellularity with loss of cell alignment and collagen birefringence was observed in the control group at week 2 (FIG. 6B). On the other hand, the graft mid-substance in the dTDSC sheet group remained intact (FIG. 6B). The cellularity in the graft mid-substance decreased at week 6. Lower cellularity and better cell alignment as well as higher collagen birefringence were observed in the graft mid-substance under bright field and polarized microscopy in the dTDSC sheet group compared to that in the control group (FIG. 6B). In both femoral and tibial tunnels, Safranin-O stainable cartilaginous region was observed at the graft-to-bone tunnel interface in the dTDSC sheet, but not in the control group, at week 2 post-ACLR (FIG. 6C). At week 6 post-ACLR, Safranin-O staining was detected at the graft-to-bone tunnel interface in both groups, with stronger staining intensity in the dTDSC sheet group (FIG. 6C).

TABLE 2

Histological scoring of graft-to-bone tunnel healing in the control group and dTDSC sheet group post-ACLR

| | Control group | dTDSC group | p value (Control group vs dTDSC sheet group) |
|---|---|---|---|
| Graft Integrity | | | |
| Femoral - Week 2 | 5 (3-5) | 5 (3-5) | n.s. |
| Femoral - Week 6 | 3 (2-3) | 4 (2-5) | n.s. |
| Tibial Epiphysis - Week 2 | 5 (3-5) | 5 (4-5) | n.s. |
| Tibial Epiphysis - Week 6 | 3 (3-4) | 5 (4-5) | ^{∗}p = 0.013 |
| Tibial Metaphysis -Week 2 | 5 (4-5) | 5 (5-5) | n.s. |
| Tibial Metaphysis -Week 6 | 2 (2-4) | 5 (4-5) | ^{∗}p = 0.013 |
| Connection Between Tendon Graft and Bone | | | |
| Femoral - Week 2 | 3 (0-4) | 5 (3-5) | ^{∗}p = 0.039 |
| Femoral - Week 6 | 5 (3-5) | 5 (4-5) | n.s. |
| Tibial Epiphysis - Week 2 | 3 (1-3) | 5 (3-5) | ^{∗}p = 0.011 |
| Tibial Epiphysis - Week 6 | 3 (3-4) | 4 (3-4) | n.s. |
| Tibial Metaphysis -Week 2 | 3 (1-3) | 5 (3-5) | ^{∗}p = 0.011 |
| Tibia Metaphysis - Week 6 | 3 (3-4) | 4 (3-4) | n.s. |
| Sharpey’s Fiber | | | |
| Femoral - Week 2 | 1 (0-1) | 2 (1-2) | ^{∗}p = 0.046 |
| Femoral - Week 6 | 3 (2-4) | 5 (3-5) | n.s. |
| Tibial Epiphysis - Week 2 | 0 (0-2) | 2 (1-5) | ^{∗}p = 0.041 |
| Tibial Epiphysis - Week 6 | 2 (1-4) | 3 (1-5) | n.s. |
| Tibial Metaphysis -Week 2 | 0 (0-2) | 2 (1-5) | ^{∗}p = 0.041 |
| Tibial Metaphysis - Week 6 | 2 (1-4) | 3 (1-5) | n.s. |
| Sum Score | | | |
| Femoral - Week 2 | 8 (5-10) | 11 (8-12) | n.s. |
| Tibial Epiphysis - Week 2 | 8 (4-9) | 11 (9-13) | ^{∗}p = 0.016 |
| Tibial Metaphysis -Week 2 | 8 (5-9) | 12 (10-13) | ^{∗∗}p = 0.008 |
| Femoral - Week 6 | 11 (7-12) | 14 (9-15) | n.s. |
| Tibial Epiphysis - Week 6 | 9 (7-10) | 11 (9-14) | n.s. |
| Tibial Metaphysis -Week 6 | 8 (6-10) | 11 (9-14) | ^{∗}p = 0.040 |
| Total - Week 2 | 21 (16-28) | 34 (30-36) | ^{∗∗}p = 0.008 |
| Total - Week 6 | 29 (23-33) | 36 (27-43) | n.s. |

The total scores of each sample were calculated as the sum of scores at different tunnel regions at different time points. The median (range) scores of each item and the median (range) of total scores at different tunnel regions at week 2 and week 6 post-ACLR were presented. * p < 0.05, **p < 0.01, n.s. not statistically significant

### Example 5—Biomechanical Testing

There was a significant improvement in the ultimate load (week 2: 116.5% increase, p=0.001; week 6: 70.4% increase, p=0.002) and stiffness (week 2: 84.7% increase, p=0.001; week 6: 79.1% increase, p=0.027) at both week 2 and week 6 in the dTDSC sheet group compared to that in the control group after ACLR (FIGS. 7A-7B). The typical force-displacement curves also indicate higher ultimate load in the dTDSC sheet group compared to the control group (FIGS. 7C-7D). At week 6, the ultimate failure load and stiffness reached 16.58 ± 7.24 N and 11.97 ± 5.21 N/mm, which was around 27.6% and 28.4% of intact rat ACL, respectively. There was no difference in the failure mode between the dTDSC sheet group and the control group at week 2 and week 6 post-ACLR (Table 3).

TABLE 3

Failure mode during biomechanical test

| Time post-ACLR | Group | Tunnel pullout | Tendon-bone junction breakage | Mid-substance failure |
|---|---|---|---|---|
| Week 2 | Control | 10 | 0 | 0 |
| | dTDSC Sheet | 9 | 1 | 0 |
| Week 6 | Control | 0 | 1 | 7 |
| | dTDSC Sheet | 0 | 0 | 10 |

Tunnel pullout: The broken graft was longer than the length of graft mid-substance (intra-articular graft), and a “hole” could be observed in either the tibia or femur. Tendon-bone junction breakage: The broken graft was of the same length as the graft mid-substance, and no “hole” could be observed in either the tibia or femur. Mid-substance failure: The broken graft was shorter than the length of graft mid-substance, and graft tissue could be observed at the intra-articular junction of the femur and tibia.

### Example 6—Immunohistochemical Staining of Inos and Cd206

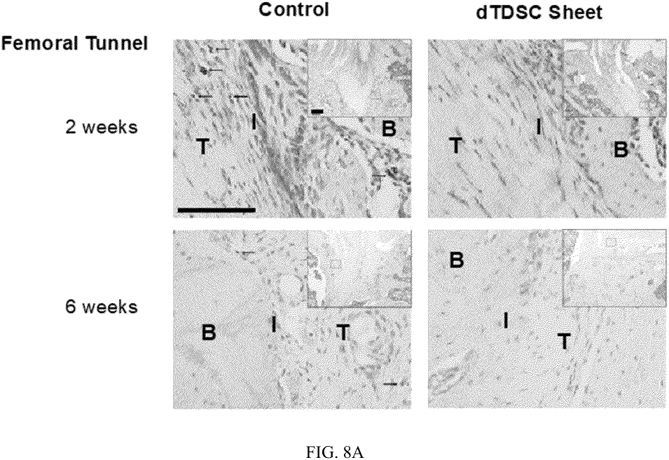

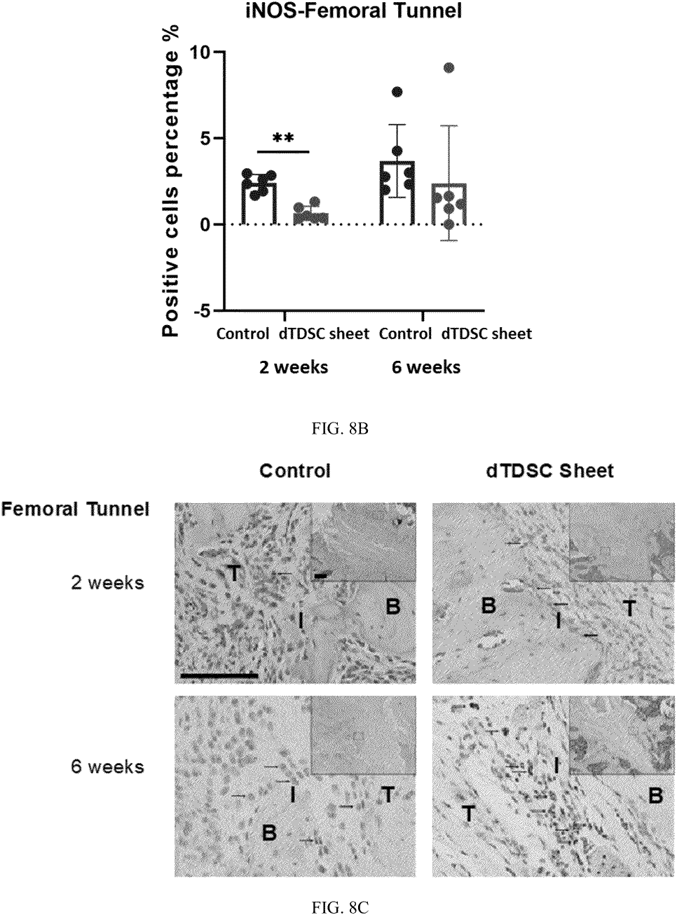

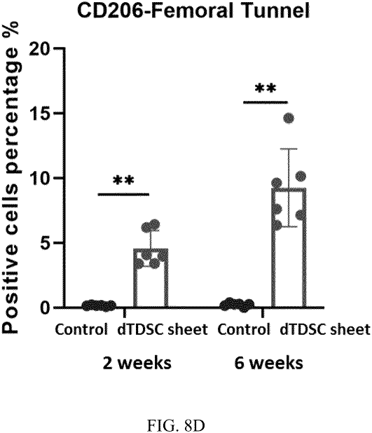

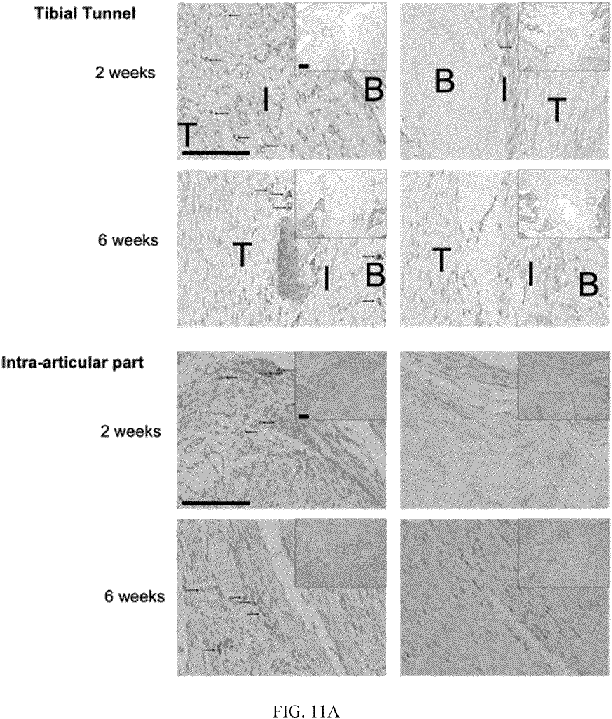

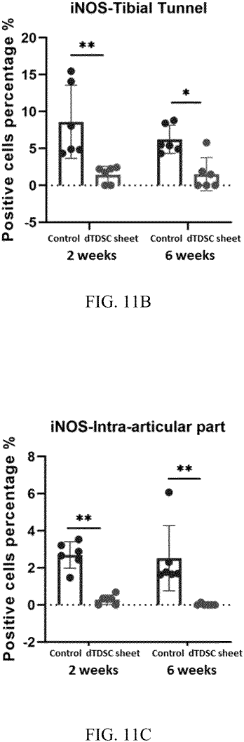

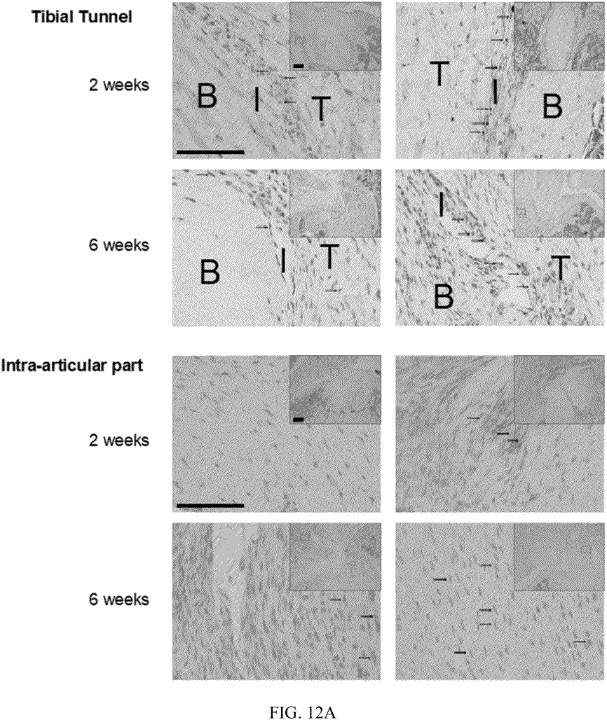

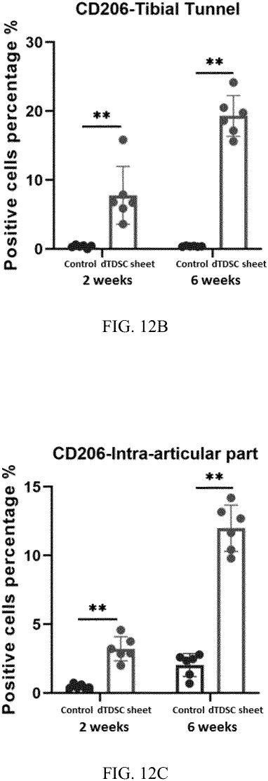

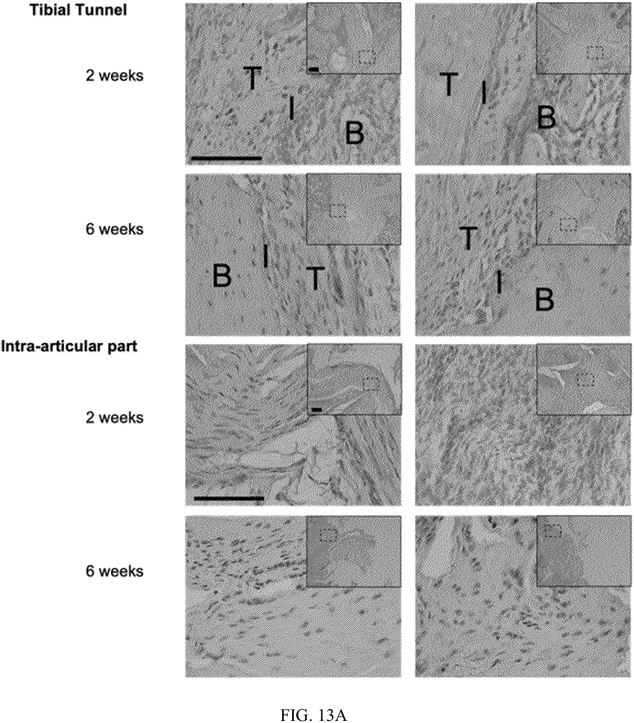

At week 2 after ACLR, there were significantly fewer iNOS⁺ cells at the graft and femoral bone tunnel interface in the dTDSC sheet group compared to the number of cells in the control group (0.65% ± 0.17% vs. 2.40% ± 0.20%, p=0.002) (FIGS. 8A-8B). On the contrary, there were significantly more CD206⁺ cells in the dTDSC sheet group compared to the number of cells in the control group at the graft and femoral bone tunnel interface (4.57% ± 0.56% vs. 0.15% ± 0.02%, p=0.002) (FIGS. 8C-8D). At week 6 post-ACLR, the number of iNOS⁺ cells was still high in the control group (3.68% ± 0.86%), but there was no significant difference compared to the number of cells in the dTDSC sheet group (2.40% ± 1.36%) (p>0.05) (FIGS. 8A-8B). The number of CD206⁺ cells reached 9.25% ± 1.23% in the dTDSC sheet group, while it remained low (0.22% ± 0.05%) in the control group at week 6 (p=0.002) (FIGS. 8C-8D). Similar results, with fewer iNOS⁺ cells and more CD206⁺ cells were observed at both the tibial tunnel and graft mid-substance in the dTDSC sheet group compared to that in the control group at week 2 and week 6 (all p<0.01) (FIGS. 11A-11C and FIGS. 12A-12C).

### Example 7—Immunohistochemical Staining of MMP/TIMP

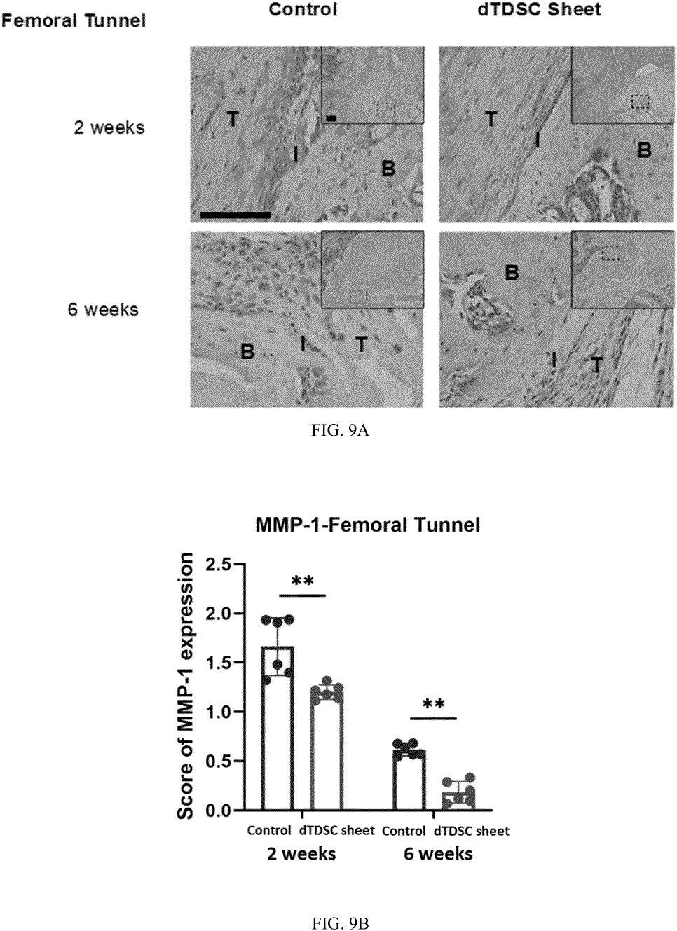

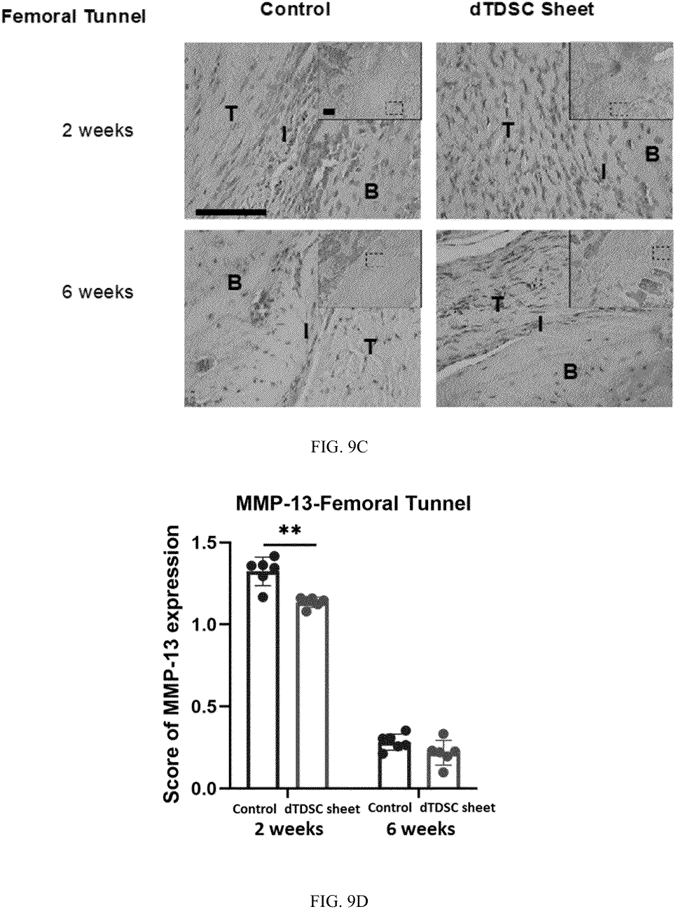

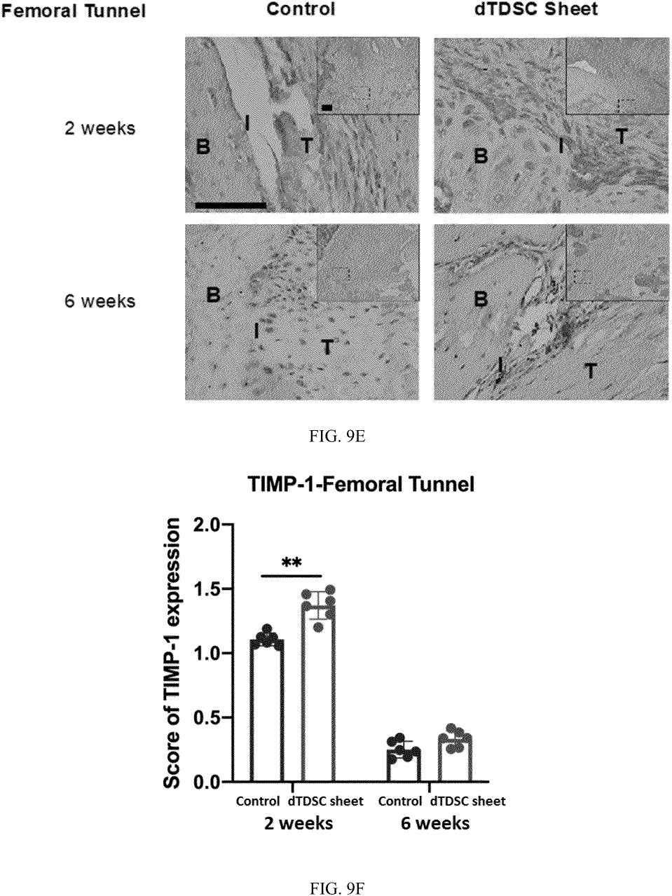

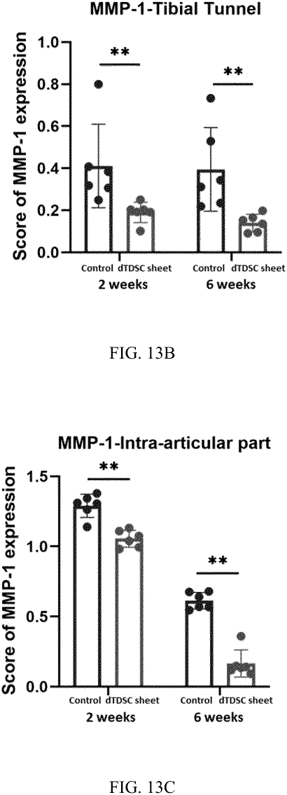

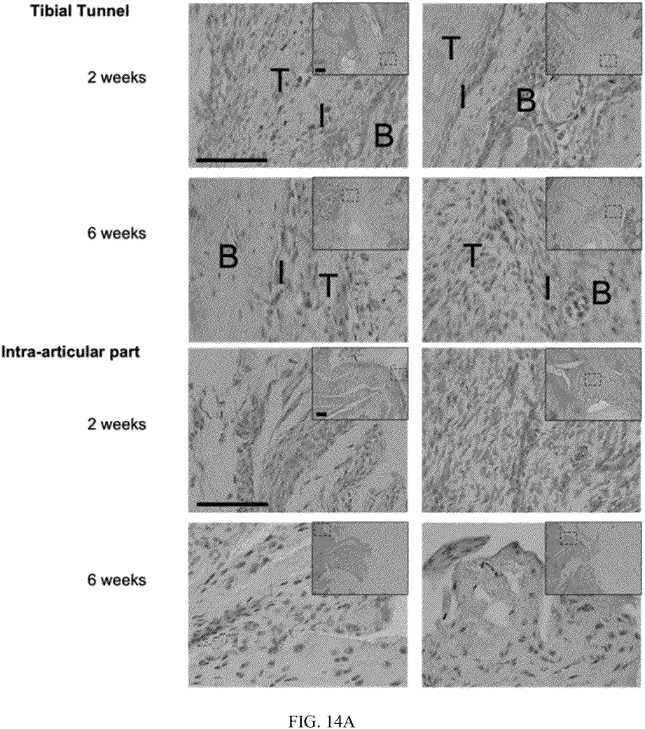

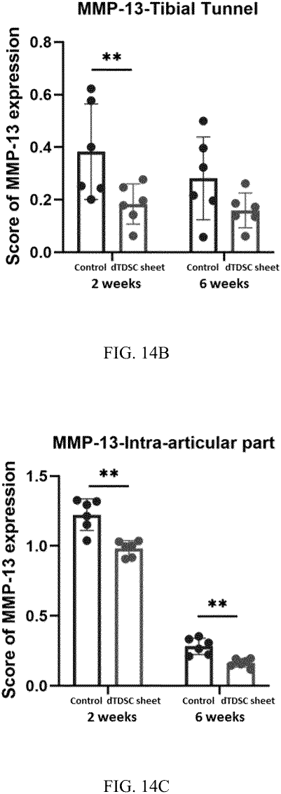

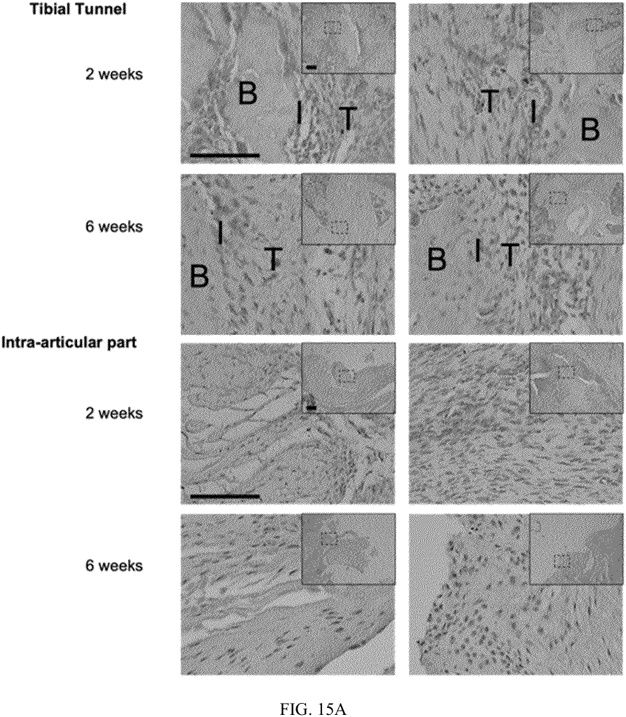

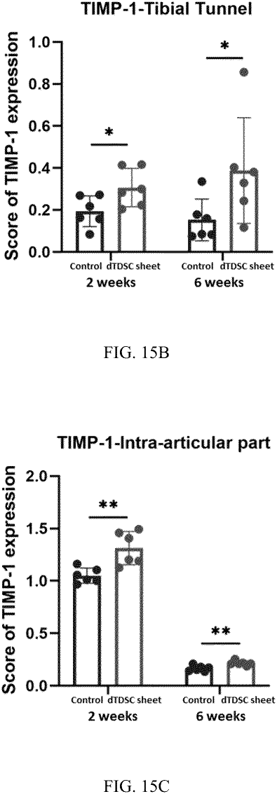

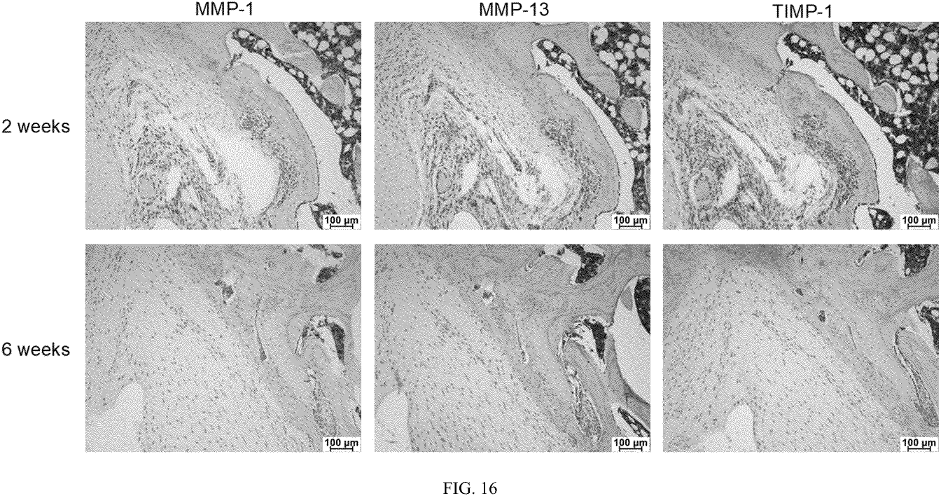

There was no expression of MMP-1, MMP-13, and TIMP-1 in the contralateral intact ACL at week 2 and week 6 post-ACLR (FIG. 16). Two weeks after ACLR, there was significantly higher expression of MMP-1 (p=0.032) (FIGS. 9A-9B) and higher expression of MMP-13 (p=0.004) (FIGS. 9C-9D) at the graft-bone tunnel interface of femoral tunnel in the control group compared to those in the dTDSC sheet group. The TIMP-1 expression at the femoral tunnel at week 2 showed an opposite result (p=0.030) (FIGS. 9E-9F). Six weeks after ACLR, the expression of MMP-1 and MMP-13 was still higher in the control group compared to that in the dTDSCs group, but there was no significant difference for the MMP-13 expression between the two groups (FIGS. 9A-9D). For the TIMP-1 expression, there was no significant difference between the two groups at week 6 (p>0.05) (FIGS. 9E-9F). Similar results were observed at the tibial tunnel interface and graft mid-substance. Except for the expression of MMP-13 at week 6, which the difference did not reach statistical significance (p>0.05), there were significant lower expression of MMP-1 and MMP-13 but higher expression of TIMP-1 at the tibial tunnel interface and graft mid-substance in the dTDSC sheet group compared to those in the control group at both week 2 and week 6 (p<0.05 or p<0.01) (FIGS. 13A-13C, FIGS. 14A-14C, and FIGS. 15A-15C).

### Example 8—Decellularization of TDSC Sheets

The subject methods for the decellularization of TDSC sheets remove 98.4% of the nucleic acid. The dTDSC sheet preserved most of the collagenous proteins, and bioactive growth factors (BMP-2, VEGF) as compared to the TDSC sheet. Tendon graft wrapped with the dTDSC sheet radiographically, histologically, and biomechanically promoted early graft healing after ACLR. There was lower expression of iNOS⁺ cells, MMP-1, and MMP-13 but higher expression of CD206⁺ cells and TIMP-1 at the tunnel interface and graft mid-substance in the dTDSC sheet group compared to those in the control group. There was no significant difference in the stiffness of the reconstructed ACL complex at week 6 in the dTDSC sheet group compared to that in the TDSC sheet group using the same animal model, suggesting that the outcome of the dTDSC sheet was not inferior to the previous study³⁷. The dTDSC sheet was less than 6 µm thick. Wrapping the tendon graft with one layer of dTDSC sheet only changed the graft diameter by less than 0.6%. The improvement in biomechanical test can be explained by the biological effects of the dTDSC sheet.

There was no observable difference in graft healing at different tunnel regions as shown by microCT imaging and histological scoring. Similar results were observed in the previous studies using the same rat model³⁵⁻³⁷. However, the previous paper using a rabbit model showed that graft healing in the tibial tunnel was inferior to that in the femoral tunnel at the tendon-to-bone interface after ACLR⁵⁸. The difference in femoral and tibial tunnel healing may be due to different bone microarchitectures of the two regions. Tendon graft to bone tunnel healing depends on the cancellous bone quantity, quality, and distribution. The femoral tunnel has more cancellous bone, which allows more rapid and stable attachment of the tendon graft²¹. Besides, MRI studies have shown that inflammatory synovial fluid was prone to enter the tibial tunnel, which might impair graft healing^{7,9}. This discrepancy may be due to the use of animal models of different species.

Similar to the TDSC sheet, the dTDSC sheet expressed bioactive factors and showed proven effects on tissue repair. No external scaffold is needed for transplantation. It supports homogeneous delivery of bioactive factors and is compatible with the arthroscopy assisted ACLR. The dTDSC sheet can be fabricated into different sizes and shapes as well as prepared from surgical waste tendon material. In addition, the use of the dTDSC sheet for augmenting ACLR eliminates the need for cell transplantation, which is complicated with issues of maintaining viability, stability, potential uncontrolled actions of transplanted cells, and high manufacturing cost. The dTDSC sheet further improves the reproducibility of the manufacturing process. The logistics for transporting the decellularized cell sheet from a Good Manufacturing Practice (GMP) facility to the operation theatre is expected to be easier. These properties facilitate the clinical translatability of the dTDSC sheet for tissue repair.

Stem cell sheets prepared from different cell sources have been applied for the promotion of graft healing after ACLR^{25,39,41}. ACL-derived CD34⁺ cell sheets enhanced graft healing by increasing proprioceptive recovery and graft maturation⁴¹. The same group later showed that BMP-2 transduced ACL-derived CD34⁺ cells further improved graft osteointegration and tensile strength of the reconstructed complex²⁵. Cell sheets formed by adipose-derived stromal cells (ADSCs)³⁹ were also reported to improve the biomechanical strength of the reconstructed complex at the early stage after ACLR. Although these cell sheets all showed regenerative capacity, ligament-derived stem cell (LDSC) / TDSC showed high proliferation, clonogenicity, and multi-lineage differentiation potential, including ligamentous / tenogenic differentiation potential, and hence may have a better potential for tendon and tendon-bone junction repair⁵². TDSCs can be isolated from the residual graft tissue during ACLR while the ACL-derived CD34⁺ cells need to be extracted from the ACL ruptured site, which may make the isolation difficult. In contrast to the previous studies, the TDSCs used for cell sheet formation in the subject invention were primed with biological factors to increase their tenogenic activity while maintaining their chondro-osteogenic markers³⁸.

Mounting evidence has demonstrated that the therapeutic effects of MSCs on tissue repair are mediated by modulating the inflammatory environment, enhancing cell growth, and promoting angiogenesis via paracrine factors^{2,19,28,62}. The ECM serves as a depot of immunomodulatory, chemotactic, and cellular programming factors secreted by MSCs. Previous studies have reported the retention of growth factors in the ECM after decellularization^{46,47,59,61}. The transplantation of bioactive decellularized ECM scaffolds has been shown to promote tissue repair in various studies^{40,42,56,57}. In ACL-related studies, small intestine submucosa (SIS) is the most studied ECM bioscaffold with controversial results. SIS wrapping showed no beneficial effect on the healing process in a goat ACL repair model^{44,45}. However, Liang et al.²⁷ showed that SIS hydrogel supported the growth and matrix production of ACL fibroblasts. In addition, Fisher et al. also reported biomechanical improvements in a goat ACL repair model after injection of SIS hydrogel¹⁷. In addition to SIS, bovine connective tissue-derived ECM⁴³ was reported to enhance ACL repair with improvement in biomechanical strength. The transplantation of physically modified tendon ECM was also reported to enhance bone and fibrocartilage formation in a rabbit ACLR model³⁰.

Depending on the composition and structural organization of tissues, the most effective method for decellularization (physical, chemical, and biological treatments and their combinations) varies¹⁰. In the subject invention, we have optimized the protocol for the decellularization of the TDSC sheet, which successfully removed the cellular components as well as preserved the collagenous proteins and bioactive factors. Triton X-100 disrupts DNA-protein interactions, lipid-lipid interactions, and lipid-protein interactions but leaves protein-protein interactions intact⁴⁹ and hence may help to preserve the ECM and collagenous proteins in the dTDSC sheet. It is less damaging to the tissue structure than ionic surfactants, and was reported to preserve the microstructure and mechanical strength of skeletal muscle tissue after decellularization²⁹. Our unpublished results showed that bone marrow-derived stromal cells (BMSCs) seeded on dTDSC sheet showed higher proliferation and migration, suggesting that the residual Triton X-100, if any, is likely non-toxic to cells. In fact, Triton X-100 is added in the manufacturing of influenza vaccines to inhibit aggregation and precipitation of biomolecules²³. There was a significant reduction in the non-collagenous proteins (71.2%) in the dTDSC sheet as shown by Fast Green FCF staining that can be due to the removal of cellular components from the TDSC sheet.

Our results showed that the dTDSC sheet preserved BMP-2 and VEGF, the administration of which has been shown to augment graft healing after ACLR^{8,11}. The improvement in tunnel bone formation, graft osteointegration, and graft mid-substance integrity in the subject invention can be mediated by the growth factors retained in the dTDSC sheet. The higher integrity of the graft can be due to the physical protection of the tendon graft by the dTDSC sheet from the damage of the inflammatory cytokines and proteases in the synovial fluid. In addition, the lower expression of MMPs and higher expression of TIMP-1 at the tendon-to-bone interface and intra-articular graft mid-substance after ACLR indicated the immunomodulatory effects of the dTDSC sheet. MMPs is a family of calcium-dependent zinc-containing endopeptidases, which can degrade collagen and other ECM components. They are inactivated by TIMPs. The inhibition of MMP activities has been reported to improve tendon-to-bone healing in the previous studies^{3,4,12}. Macrophages are major producers of MMPs under inflammatory situations¹⁵. Our data shows higher expression of MMP-1 and MMP-13 in regions accumulated with M1 macrophages but higher expression of TIMP-1 in areas dominated by M2 macrophages. The infiltration of M1 macrophages was positively correlated with graft loosening in the second-look arthroscopy in the first year after ACLR⁵⁰. Higher expression of M1 macrophages, MMP-1, and MMP-13 at the peri-tunnel region were also associated with greater peri-tunnel bone loss at the tibia after ACLR in a rat model³⁵. The polarization of pro-inflammatory M1 macrophages to the regenerative M2 macrophages was associated with better tendon and ligament healing in the previous studies^{31,51}. MSCs can polarize macrophages to M2 immunophenotype via paracrine mechanisms^{5,20,26}. Membrane particles isolated from MSCs were shown to bind to and induce selective apoptosis of pro-inflammatory CD14⁺CD16⁺ monocytes²⁰. Besides, BMSCs-treated macrophages were reported to reduce the expression of pro-inflammatory factors and increase the expression of M2 markers compared to the untreated macrophages⁵. Our results therefore suggested that the dTDSC sheet can promote graft healing by enhancing bone formation, suppressing inflammation, reducing matrix degradation, promoting angiogenesis, and physically protecting the graft mid-substance.

An optimized protocol for the decellularization of the TDSC sheet was developed. The dTDSC sheet exhibited a similar gross morphology, comparable levels of collagenous proteins, and bioactive growth factors (BMP-2 and VEGF) to the TDSC sheet. Wrapping tendon graft with dTDSC sheet promoted graft healing after ACLR, likely via enhancing bone formation and angiogenesis, modulating macrophage polarization and MMP/TIMP expression, and physically protecting the tendon graft. dTDSC sheets alleviate the quality control and safety concerns of cell transplantation and may be used as a cell-free alternative for the promotion of graft healing in ACLR.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Awad HA, Boivin GP, Dressler MR, et al. Repair of patellar tendon injuries using a cell-collagen composite. Journal of Orthopaedic Research. 2003;21(3):420-431.

2. Baraniak PR, McDevitt TC. Stem cell paracrine actions and tissue regeneration. Regen Med. 2010;5(1):121-143.

3. Bedi A, Fox AJ, Kovacevic D, et al. Doxycycline-mediated inhibition of matrix metalloproteinases improves healing after rotator cuff repair. The American journal of sports medicine. 2010;38(2):308-317.

4. Bedi A, Kovacevic D, Hettrich C, et al. The effect of matrix metalloproteinase inhibition on tendon-to-bone healing in a rotator cuff repair model. J Shoulder Elbow Surg. 2010;19(3):384-391.

5. Bouchlaka MN, Moffitt AB, Kim J, et al. Human Mesenchymal Stem Cell-Educated Macrophages Are a Distinct High IL-6-Producing Subset that Confer Protection in Graft-versus-Host-Disease and Radiation Injury Models. Biol Blood Marrow Transplant. 2017;23(6):897-905.

6. Breitbach M, Bostani T, Roell W, et al. Potential risks of bone marrow cell transplantation into infarcted hearts. Blood. 2007;110(4):1362-1369.

7. Buelow JU, Siebold R, Ellermann A. A prospective evaluation of tunnel enlargement in anterior cruciate ligament reconstruction with hamstrings: extracortical versus anatomical fixation. Knee Surg Sports Traumatol Arthrosc. 2002;10(2):80-85.

8. Chen J, Yang L, Guo L, Duan X. Sodium hyaluronate as a drug-release system for VEGF 165 improves graft revascularization in anterior cruciate ligament reconstruction in a rabbit model. Experimental and therapeutic medicine. 2012;4(3):430-434.

9. Clatworthy MG, Annear P, Bulow JU, Bartlett RJ. Tunnel widening in anterior cruciate ligament reconstruction: a prospective evaluation of hamstring and patella tendon grafts. Knee Surg Sports Traumatol Arthrosc. 1999;7(3):138-145.

10. Crapo PM, Gilbert TW, Badylak SF. An overview of tissue and whole organ decellularization processes. Biomaterials. 2011;32(12):3233-3243.

11. Crispim JF, Fu SC, Lee YW, et al. Bioactive Tape With BMP-2 Binding Peptides Captures Endogenous Growth Factors and Accelerates Healing After Anterior Cruciate Ligament Reconstruction. Am J Sports Med. 2018;46(12):2905-2914.

12. Demirag B, Sarisozen B, Ozer O, Kaplan T, Ozturk C. Enhancement of tendon-bone healing of anterior cruciate ligament grafts by blockage of matrix metalloproteinases. The Journal of bone and joint surgery American volume. 2005;87(11):2401-2410.

13. Desgres M, Menasche P. Clinical Translation of Pluripotent Stem Cell Therapies: Challenges and Considerations. Cell stem cell. 2019;25(5):594-606.

14. Ekdahl M, Wang JH, Ronga M, Fu FH. Graft healing in anterior cruciate ligament reconstruction. Knee Surg Sports Traumatol Arthrosc. 2008;16(10):935-947.

15. Elkington PT, Green JA, Friedland JS. Analysis of matrix metalloproteinase secretion by macrophages. Methods Mol Biol. 2009;531:253-265.

16. Faulk DM, Johnson SA, Badylak SF. Decellularized biological scaffolds for cardiac repair and regeneration. In: Cardiac Regeneration and Repair. 2014:180-200.

17. Fisher MB, Liang R, Jung HJ, et al. Potential of healing a transected anterior cruciate ligament with genetically modified extracellular matrix bioscaffolds in a goat model. Knee surgery, sports traumatology, arthroscopy : official journal of the ESSKA. 2012;20(7):1357-1365.

18. Gilpin A, Yang Y. Decellularization Strategies for Regenerative Medicine: From Processing Techniques to Applications. BioMed research international. 2017;2017:9831534.

19. Gnecchi M, Zhang Z, Ni A, Dzau VJ. Paracrine mechanisms in adult stem cell signaling and therapy. Circ Res. 2008;103(11):1204-1219.

20. Goncalves FDC, Luk F, Korevaar SS, et al. Membrane particles generated from mesenchymal stromal cells modulate immune responses by selective targeting of pro-inflammatory monocytes. Scientific reports. 2017;7(1):12100.

21. Grassman SRM, McDonald DB, Thornton GM, Shrive NG, Frank CB. Early healing processes of free tendon grafts within bone tunnels is bone-specific: a morphological study in a rabbit model. The Knee. 2002;9(1):21-26.

22. Harris MT, Butler DL, Boivin GP, et al. Mesenchymal stem cells used for rabbit tendon repair can form ectopic bone and express alkaline phosphatase activity in constructs. J Orthop Res. 2004;22(5):998-1003.

23. Heinig K, Vogt C. Determination of Triton X-100 in influenza vaccine by high-performance liquid chromatography and capillary electrophoresis. Fresenius’ Journal of Analytical Chemistry. 1997;359(2):202-206.

24. Hexter AT, Thangarajah T, Blunn G, Haddad FS. Biological augmentation of graft healing in anterior cruciate ligament reconstruction: a systematic review. Bone Joint J. 2018;100-B(3):271-284.

25. Kawakami Y, Takayama K, Matsumoto T, et al. Anterior Cruciate Ligament-Derived Stem Cells Transduced With BMP2 Accelerate Graft-Bone Integration After ACL Reconstruction. Am J Sports Med. 2017;45(3):584-597.

26. Kim J, Hematti P. Mesenchymal stem cell-educated macrophages: a novel type of alternatively activated macrophages. Experimental hematology. 2009;37(12):1445-1453.

27. Liang R, Yang G, Kim KE, et al. Positive effects of an extracellular matrix hydrogel on rat anterior cruciate ligament fibroblast proliferation and collagen mRNA expression. Journal of orthopaedic translation. 2015;3(3):114-122.

28. Liang X, Ding Y, Zhang Y, Tse HF, Lian Q. Paracrine mechanisms of mesenchymal stem cell-based therapy: current status and perspectives. Cell transplantation. 2014;23(9):1045-1059.

29. Lin CH, Yang JR, Chiang NJ, Ma H, Tsay RY. Evaluation of decellularized extracellular matrix of skeletal muscle for tissue engineering. Int J Artif Organs. 2014;37(7):546-555.

30. Liu H, Yang L, Zhang E, et al. Biomimetic tendon extracellular matrix composite gradient scaffold enhances ligament-to-bone junction reconstruction. Acta biomaterialia. 2017;56:129-140.

31. Liu J, Chen B, Bao J, et al. Macrophage polarization in periodontal ligament stem cells enhanced periodontal regeneration. Stem Cell Res Ther. 2019;10(1):320.

32. Lopez-De Leon A, Rojkind M. A simple micromethod for collagen and total protein determination in formalin-fixed paraffin-embedded sections. J Histochem Cytochem. 1985;33(8):737-743.

33. Lui PP. A practical guide for the isolation and maintenance of stem cells from tendon. Methods Mol Biol. 2015;1212:127-140.

34. Lui PP, Ho G, Lee YW, et al. Validation of a histologic scoring system for the examination of quality of tendon graft to bone tunnel healing in anterior cruciate ligament reconstruction. Anal Quant Cytol Histol. 2011;33(1):36-49.

35. Lui PP, Lee YW, Mok TY, Cheuk YC. Peri-tunnel bone loss: does it affect early tendon graft to bone tunnel healing after ACL reconstruction? Knee surgery, sports traumatology, arthroscopy : official journal of the ESSKA. 2015;23(3):740-751.

36. Lui PP, Lee YW, Mok TY, Cheuk YC, Chan KM. Alendronate reduced peri-tunnel bone loss and enhanced tendon graft to bone tunnel healing in anterior cruciate ligament reconstruction. European cells & materials. 2013;25:78-96.

37. Lui PP, Wong OT, Lee YW. Application of tendon-derived stem cell sheet for the promotion of graft healing in anterior cruciate ligament reconstruction. Am J Sports Med. 2014;42(3):681-689.

38. Lui PP, Wong OT, Lee YW. Transplantation of tendon-derived stem cells pretreated with connective tissue growth factor and ascorbic acid in vitro promoted better tendon repair in a patellar tendon window injury rat model. Cytotherapy. 2016;18(1):99-112.

39. Matsumoto T, Sato Y, Kobayashi T, et al. Adipose-Derived Stem Cell Sheets Improve Early Biomechanical Graft Strength in Rabbits After Anterior Cruciate Ligament Reconstruction. Am J Sports Med. 2021:3635465211041582.

40. Mertsch S, Hasenzahl M, Reichl S, Geerling G, Schrader S. Decellularized human corneal stromal cell sheet as a novel matrix for ocular surface reconstruction. Journal of tissue engineering and regenerative medicine. 2020;14(9):1318-1332.

41. Mifune Y, Matsumoto T, Takayama K, et al. Tendon graft revitalization using adult anterior cruciate ligament (ACL)-derived CD34+ cell sheets for ACL reconstruction. Biomaterials. 2013;34(22):5476-5487.

42. Mohiuddin OA, Campbell B, Poche JN, et al. Decellularized Adipose Tissue: Biochemical Composition, in vivo Analysis and Potential Clinical Applications. Advances in experimental medicine and biology. 2020;1212:57-70.

43. Murray MM, Fleming BC. Use of a bioactive scaffold to stimulate anterior cruciate ligament healing also minimizes posttraumatic osteoarthritis after surgery. Am J Sports Med. 2013;41(8):1762-1770.

44. Nguyen DT, Dellbrugge S, Tak PP, et al. Histological characteristics of ligament healing after bio-enhanced repair of the transected goat ACL. J Exp Orthop. 2015;2(1):4.

45. Nguyen DT, Geel J, Schulze M, et al. Healing of the goat anterior cruciate ligament after a new suture repair technique and bioscaffold treatment. Tissue engineering Part A. 2013;19(19-20):2292-2299.

46. Ning LJ, Jiang YL, Zhang CH, et al. Fabrication and characterization of a decellularized bovine tendon sheet for tendon reconstruction. J Biomed Mater Res A. 2017;105(8):2299-2311.

47. Ning LJ, Zhang Y, Chen XH, et al. Preparation and characterization of decellularized tendon slices for tendon tissue engineering. J Biomed Mater Res A. 2012;100(6):1448-1456.

48. Roach HI, Mehta G, Oreffo RO, Clarke NM, Cooper C. Temporal analysis of rat growth plates: cessation of growth with age despite presence of a physis. J Histochem Cytochem. 2003;51(3):373-383.

49. Seddon AM, Curnow P, Booth PJ. Membrane proteins, lipids and detergents: not just a soap opera. Biochim Biophys Acta. 2004;1666(1-2):105-117.

50. Song B, Jiang C, Luo H, et al. Macrophage M1 Plays a Positive Role in Aseptic Inflammation-Related Graft Loosening After Anterior Cruciate Ligament Reconstruction Surgery. Inflammation. 2017;40(6):1815-1824.

51. Sunwoo JY, Eliasberg CD, Carballo CB, Rodeo SA. The role of the macrophage in tendinopathy and tendon healing. J Orthop Res. 2020;38(8):1666-1675.

52. Tan Q, Lui PP, Rui YF, Wong YM. Comparison of potentials of stem cells isolated from tendon and bone marrow for musculoskeletal tissue engineering. Tissue engineering Part A. 2012;18(7-8):840-851.

53. Tasso R, Augello A, Carida M, et al. Development of sarcomas in mice implanted with mesenchymal stem cells seeded onto bioscaffolds. Carcinogenesis. 2009;30(1):150-157.

54. Varghese F, Bukhari AB, Malhotra R, De A. IHC Profiler: an open source plugin for the quantitative evaluation and automated scoring of immunohistochemistry images of human tissue samples. PloS one. 2014;9(5):e96801.

55. Wang C, Hu Y, Zhang S, et al. Application of Stem Cell Therapy for ACL Graft Regeneration. Stem cells international. 2021;2021:6641818.

56. Wang Z, Han L, Sun T, et al. Extracellular matrix derived from allogenic decellularized bone marrow mesenchymal stem cell sheets for the reconstruction of osteochondral defects in rabbits. Acta Biomater. 2020;118:54-68.

57. Wang Z, Li Z, Li Z, et al. Cartilaginous extracellular matrix derived from decellularized chondrocyte sheets for the reconstruction of osteochondral defects in rabbits. Acta biomaterialia. 2018;81:129-145.

58. Wen CY, Qin L, Lee KM, Wong MW, Chan KM. Grafted tendon healing in tibial tunnel is inferior to healing in femoral tunnel after anterior cruciate ligament reconstruction: a histomorphometric study in rabbits. Arthroscopy : the journal of arthroscopic & related surgery : official publication of the Arthroscopy Association of North America and the International Arthroscopy Association. 2010;26(1):58-66.

59. Xing Q, Yates K, Tahtinen M, et al. Decellularization of fibroblast cell sheets for natural extracellular matrix scaffold preparation. Tissue engineering Part C, Methods. 2015;21(1):77-87.

60. Yao S, Fu BS, Yung PS. Graft healing after anterior cruciate ligament reconstruction (ACLR). Asia Pac J Sports Med Arthrosc Rehabil Technol. 2021;25:8-15.

61. Zhou S, Wang Y, Zhang K, et al. The Fabrication and Evaluation of a Potential Biomaterial Produced with Stem Cell Sheet Technology for Future Regenerative Medicine. Stem Cells Int. 2020;2020:9567362.

62. Zhou Y, Yamamoto Y, Xiao Z, Ochiya T. The Immunomodulatory Functions of Mesenchymal Stromal/Stem Cells Mediated via Paracrine Activity. Journal of clinical medicine. 2019;8(7).

63. Rui YF, Lui PP, Ni M, Chan LS, Lee YW, Chan KM. Mechanical loading increased BMP-2 expression which promoted osteogenic differentiation of tendon-derived stem cells. J Orthop Res. 2011;29(3):390-396.

64. Rui YF, Lui PP, Li G, Fu SC, Lee YW, Chan KM. Isolation and characterization of multipotent rat tendon-derived stem cells. Tissue Eng Part A. 2010 May;16(5):1549-58.

We claim:

1. A decellularized stem cell sheet comprising extracellular matrix (ECM), small RNA molecules, and bioactive factors secreted by stem cells.

2. The decellularized stem cell sheet of claim 1, wherein the stem cells are isolated from animal or human tissues.

3. The decellularized stem cell sheet of claim 2, wherein the stem cells are adult stem cells derived from a tendon, ligament, bone marrow, adipose tissue, umbilical cord blood, or dental pulp.

4. The decellularized stem cell sheet of claim 1, further comprising collagenous and non-collagenous proteins.

5. The decellularized stem cell sheet of claim 1, wherein the decellularized stem cell sheet does not contain cells or dsDNA.

6. The decellularized stem cell sheet of claim 1, wherein the small RNA are less than about 200 base pairs in length.

7. The decellularized stem cell sheet of claim 1, wherein the bioactive factors secreted by stem cells are bone morphogenetic protein-2 (BMP-2) and vascular endothelial growth factor (VEGF).

8. The decellularized stem cell sheet of claim 1, wherein the thickness of the decellularized stem cell sheet is less than about 6 µm.

9. A method of decellularizing a stem cell sheet, the method comprising:
(i) isolating a stem cell;
(ii) culturing the stem cell under conditions that form a stem cell sheet;
(iii) incubating the stem cell sheet with a decellularization solution to the stem cell sheet, wherein the decellularization solution comprises about 0.05% to about 0.5% Triton X-100, 1 mM to about 20 mM Tris, about 10 to about 40 mM EDTA, and about 0.5 µg/mL to about 3 µg/mL aprotinin; and
(iv) treating the stem cell sheet with DNase I.

10. The method of claim 9, wherein the decellularization solution comprises about 0.3% Triton X-100, about 25 mM EDTA, about 10 mM Tris, and about 1 µg/mL aprotinin.

11. The method of claim 9, further comprising rinsing the stem cell sheet with a buffer after (ii), (iii) (iv), or any combination thereof.

12. The method of claim 11, wherein the buffer is PBS or NaCl and the rinsing occurs at about 4° C. to about 37° C. for about 1 minute to about 36 hours.

13. A method of repairing musculoskeletal and/or connective tissues in a subject, the method comprising applying a decellularized stem cell sheet to musculoskeletal and/or connective tissue where tissue repair is required, wherein the decellularized stem cell sheet comprises ECM, small RNA molecule, and bioactive factors secreted by stem cells.

14. The method of claim 13, wherein the decellularized stem cell sheet comprises collagenous and non-collagenous proteins.

15. The method of claim 13, wherein the decellularized stem cell sheet does not contain cells or dsDNA.

16. The method of claim 13, wherein the small RNA are less than about 200 base pairs in length.

17. The method of claim 13, wherein the bioactive factors secreted by stem cells are bone morphogenetic protein-2 (BMP-2) and vascular endothelial growth factor (VEGF).

18. The method of claim 13, wherein the subject is a mammal.

19. The method of claim 13, wherein the subject is a human.

20. The method of claim 13, wherein the musculoskeletal and/or connective tissue is tendon, ligament, bone, cartilage, muscle, or skin.

21. The method of claim 20, wherein the tendon in need of repair is at the tendon-bone junction.

22. The method of claim 21, wherein the repair at the tendon-bone junction is a tendon graft to bone tunnel healing and graft remodeling in ACL reconstruction, rotator cuff repair, or patellar bone-patellar tendon repair.

23. The method of claim 20, wherein the tendon in need of repair is a tendon window defect.

24. The method of claim 20, wherein the tendon or ligament in need of repair is a result of a tendon or ligament rupture.

25. The method of claim 20, wherein the bone in need of repair is fractured.

26. The method of claim 20, wherein the cartilage in need of repair results from osteoarthritis or has a osteo-chondro defect.

27. The method of claim 20, wherein the muscle in need of repair comprises a muscle tear.

28. The method of claim 20, wherein the skin in need of repair is wounded or burned.

29. A method of synthesis of bio-artificial tissue in a subject, the method comprising:
i) layering a decellularized stem cell sheet on at least one type of biomaterial, wherein the decellularized stem cell sheet comprises ECM, small RNA molecule, and bioactive factors secreted by stem cells; and
ii) seeding the layered decellularized stem cell sheet with stem cells.

30. The method of claim 29, further comprising:
iii) rolling and mechanically loading the seeded decellularized stem cell sheet to a site in need of musculoskeletal tissue repair.

31. The method of claim 29, wherein the decellularized stem cell sheet provides a scaffold for cells or growth factors.

32. The method of claim 29, wherein the decellularized stem cell sheet provides a scaffold for synthesis of bio-artificial tendon and ligament graft tissue for tendon/ligament replacement.

33. The method of claim 29, wherein the biomaterial is polylactic acid (PLA) or poly(lactic-co-glycolic acid) (PLGA).

34. The method of claim 29, wherein the stem cells are TDSC, adipose tissue-derived stromal cells (ADSC), bone marrow-derived stromal cells (BMSC), or umbilical cord blood stromal cells.

35. The method of claim 29, wherein the small RNA molecules are less than about 200 base pairs in length.

36. The method of claim 29, wherein the bioactive factors secreted by stem cells are BMP-2 and vascular endothelial growth factor VEGF.

37. The method of claim 29, wherein the decellularized stem cell sheet comprises collagenous and non-collagenous proteins.

38. The method of claim 29, wherein the decellularized stem cell sheet does not contain cells or dsDNA.
